SEQUENCE LISTING (53 sequences)

SEQ ID NO: 1 - DNA, 1348 bp, Homo sapiens
  CDS (102)...(980)
ggggacaattcagaggctgctgcctgcttaggaggttgtagaaagctctgtaggttctctctgtgtgtcctacaggagtcttcaggccagctccctgtcggatggcttttatgaaaaaatatctcctccccattctggggctcttcatggcctactactactattctgcaaacgaggaattcagaccagagatgctccaaggaaagaaagtgattgtcacaggggccagcaaagggatcggaagagagatggcttatcatctggcgaagatgggagcccatgtggtggtgacagcgaggtcaaaagaaactctacagaaggtggtatcccactgcctggagcttggagcagcctcagcacactacattgctggcaccatggaagacatgaccttcgcagagcaatttgttgcccaagcaggaaagctcatgggaggactagacatgctcattctcaaccacatcaccaacacttctttgaatctttttcatgatgatattcaccatgtgcgcaaaagcatggaagtcaacttcctcagttacgtggtcctgactgtagctgccttgcccatgctgaagcagagcaatggaagcattgttgtcgtctcctctctggctgggaaagtggcttatccaatggttgctgcctattctgcaagcaagtttgctttggatgggttcttctcctccatcagaaaggaatattcagtgtccagggtcaatgtatcaatcactctctgtgttcttggcctcatagacacagaaacagccatgaaggcagtttctgggatagtccatatgcaagcagctccaaaggaggaatgtgccctggagatcatcaaagggggagctctgcgccaggaagaagtgtattatgacagctcactctggaccactcttctgatcagaaatccatgcaggaagatcctggaatttctctactcaacgagctataatatggacagattcataaacaagtaggaactccctgagggctgggcatgctgagggattttgggactgttctgtctcatgtttatctgagctcttatctatgaagacatcttcccagagtgtccccagagacatgcaagtcatgggtcacacctgacaaatggaaggagttcctctaacatttgcaaaatggaaatgtaataataatgaatgtcatgcaccgctgcagccagcagttgtaaaattgttagtaaacataggtataattaccagatagttatattaaatttatatcttatatataataatatgtgatgattaatacaatattaattataataaaggtcacataaactttataaatccaaaaaaaaaaaaaaaaaaaaaaaaaaaaa

SEQ ID NO: 2 - protein, 292 aa, Homo sapiens
MAFMKKYLLPILGLFMAYYYYSANEEFRPEMLQGKKVIVTGASKGIGREMAYHLAKMGAHVVVTARSKETLQKVVSHCLELGAASAHYIAGTMEDMTFAEQFVAQAGKLMGGLDMLILNHITNTSLNLFHDDIHHVRKSMEVNFLSYVVLTVAALPMLKQSNGSIVVVSSLAGKVAYPMVAAYSASKFALDGFFSSIRKEYSVSRVNVSITLCVLGLIDTETAMKAVSGIVHMQAAPKEECALEIIKGGALRQEEVYYDSSLWTTLLIRNPCRKILEFLYSTSYNMDRFINK

SEQ ID NO: 3 - DNA, 2478 bp, Homo sapiens
  CDS (29)...(2227)
agctgcaggactctgctctcctgcggccatgggccagggttgggctactgcaggacttcccagcctcctcttcctgctgctctgctacgggcaccctctgctggtccctagccaggaggcatcccaacaggtgacagtcacccatgggacaagcagccaggcaacaaccagcagccagacaaccacccaccaggcgacggcccaccagacatcagcccagagcccaaacctggtgactgatgaggctgaggccagcaagtttgtggaggaatatgaccggacatcccaggtggtgtggaacgagtatgccgaggccaactggaactacaacaccaacatcaccacagagaccagcaagattctgctgcagaagaacatgcaaatagccaaccacaccctgaagtacggcacccaggccaggaagtttgatgtgaaccagttgcagaacaccactatcaagcggatcataaagaaggttcaggacctagaacgggcagcgctgcctgcccaggagctggaggagtacaacaagatcctgttggatatggaaaccacctacagcgtggccactgtgtgccacccgaatggcagctgcctgcagctcgagccagatctgacgaatgtgatggccacatcccggaaatatgaagacctgttatgggcatgggagggctggcgagacaaggcggggagagccatcctccagttttacccgaaatacgtggaactcatcaaccaggctgcccggctcaatggctatgtagatgcaggggactcgtggaggtctatgtacgagacaccatccctggagcaagacctggagcggctcttccaggagctgcagccactctacctcaacctgcatgcctacgtgcgccgggccctgcaccgtcactacggggcccagcacatcaacctggaggggcccattcctgctcacctgctggggaacatgtgggcgcagacctggtccaacatctatgacttggtggtgcccttcccttcagccccctcgatggacaccacagaggctatgctaaagcagggctggacgcccaggaggatgtttaaggaggctgatgatttcttcacctccctggggctgctgcccgtgcctcctgagttctggaacaagtcgatgctggagaagccaaccgacgggcgggaggtggtctgccacgcctcggcctgggacttctacaacggcaaggacttccggatcaagcagtgcaccaccgtgaacttggaggacctggtggtggcccaccacgaaatgggccacatccagtatttcatgcagtacaaagacttacctgtggccttgagggagggtgccaaccccggcttccatgaggccattggggacgtgctagccctctcagtgtctacgcccaagcacctgcacagtctcaacctgctgagcagtgagggtggcagcgacgagcatgacatcaactttctgatgaagatggcccttgacaagatcgcctttatccccttcagctacctcgtcgatcagtggcgctggagggtatttgatggaagcatcaccaaggagaactataaccaggagtggtggagcctcaggctgaagtaccagggcctctgccccccagtgcccaggactcaaggtgactttgacccaggggccaagttccacattccttctagcgtgccttacatcaggtactttgtcagcttcatcatccagttccagttccacgaggcactgtgccaggcagctggccacacgggccccctgcacaagtgtgacatctaccagtccaaggaggccgggcagcgcctggcgaccgccatgaagctgggcttcagtaggccgtggccggaagccatgcagctgatcacgggccagcccaacatgagcgcctcggccatgttgagctacttcaagccgctgctggactggctccgcacggagaacgagctgcatggggagaagctgggctggccgcagtacaactggacgccgaactccgctcgctcagaagggcccctcccagacagcggccgcgtcagcttcctgggcctggacctggatgcgcagcaggcccgcgtgggccagtggctgctgctcttcctgggcatcgccctgctggtagccaccctgggcctcagccagcggctcttcagcatccgccaccgcagcctccaccggcactcccacgggccccagttcggctccgaggtggagctgagacactcctgaggtgacccggctgggtcggccctgcccaagggcctcccaccagagactgggatgggaacactggtgggcagctgaggacacaccccacaccccagcccaccctgctcctcctgccctgtccctgtccccctcccctcccagtcctccaccaccagccgccccagccccttctcccagcacacggctgcctgacactgagccccacctctccaagtctccctgtgaatacaattaaaggtcctgccctccca

SEQ ID NO: 4 - protein, 732 aa, Homo sapiens
MGQGWATAGLPSLLFLLLCYGHPLLVPSQEASQQVTVTHGTSSQATTSSQTTTHQATAHQTSAQSPNLVTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPNSARSEGPLPDSGRVSFLGLDLDAQQARVGQWLLLFLGIALLVATLGLSQRLFSIRHRSLHRHSHGPQFGSEVELRHS

SEQ ID NO: 5 - DNA, 2051 bp, Homo sapiens
  CDS (61)...(1224)
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatctgatttagggaaactgtggcagatgagtgggagactggttgcaaggtgtgaccacaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactggggggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttcgttttaccattaaaagggaaacgtgcctctgccccacgggtagagggggtgcacgttcctcctggttccttcgcttgtgtttctgtacttaccaaaaatctaccacttcaataaattttgataggagacaaaaaaaaaa

SEQ ID NO: 6 - protein, 387 aa, Homo sapiens
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWNFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 7 - DNA, 2087 bp, Homo sapiens
  CDS (104)...(1612)
ggcacgaggagccgagcggcgaggtgcctctgtggccgcacggcaggcccgggcgacacggagacgtggagcgcgccggctcgctgcagctccgggactcaacatgcgctgctcgccgggaggcgtctggctgggcctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacactctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacagtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagcgccgtgggcccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacactggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatactccttggcacatccgtaccatcggtcagcagggccactgagtagtcattttgccattagccctcagcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaaaaaaaaa

SEQ ID NO: 8 - protein, 502 aa, Homo sapiens [sequence omitted]

SEQ ID NO: 9 - DNA, 2585 bp, Homo sapiens
  CDS (248)...(1825)
gagtcgccacgcgtccggccaccaccgccgccgccgccgctccgccagacctgctgccagcttgcccggtccagccctgagagagcctcgaacgccagctgcgagggtcatgagccagagagccccggggcgccgcgcggagagcaagcggagatagcgactttgcgccccccagccctcgccttcttgcatcgcgttccccgcatcctcgggtccttctgtcctttccgctgtccccaccgccgccatggccaccttgctccgcagcaagctgtccaacgtggccacgtccgtgtccaacaagtcccaggccaagatgagcggcatgttcgccaggatgggttttcaggcggccacggatgaggaggcggtgggcttcgcgcattgcgacgacctcgactttgagcaccgccagggcctgcagatggacatcctgaaagccgagggagagccctgcggggacgagggcgctgaagcgcccgtcgagggagacatccattatcagcgaggcagcggagctcctctgccgccctccggctccaaggaccaggtgggaggtggtggcgaattcgggggccacgacaagcccaaaatcacggcgtgggaggcaggctggaacgtgaccaacgccatccagggcatgttcgtgctgggcctaccctacgccatcctgcacggcggctacctggggttgtttctcatcatcttcgccgccgttgtgtgctgctacaccggcaagatcctcatcgcgtgcctgtacgaggagaatgaagacggcgaggtggtgcgcgtgcgggactcgtacgtggccatagccaacgcctgctgcgccccgcgcttcccaacgctgggcggccgagtggtgaacgtagcgcagatcatcgagctggtgatgacgtgcatcctgtacgtggtggtgagtggcaacctcatgtacaacagcttcccggggctgcccgtgtcgcagaagtcctggtccattatcgccacggccgtgctgctgccttgcgccttccttaagaacctcaaggccgtgtccaagttcagtctgctgtgcactctggcccacttcgtcatcaatatcctggtcatagcctactgtctatcgcgggcgcgcgactgggcctgggagaaggtcaagttctacatcgacgtcaagaagttccccatctccattggcatcatcgtgttcagctacacgtctcagatcttcctgccttcgctggagggcaatatgcagcagcccagcgagttccactgcatgatgaactggacgcacatcgcagcctgcgtgctcaagggcctcttcgcgctcgtcgcctacctcacctgggccgacgagaccaaggaggtcatcacggataacctgcccggctccatccgcgccgtggtcaacatctttctggtggccaaggcgctgttgtcctatcctctgccattctttgccgctgtcgaggtgctggagaagtcgctcttccaggaaggcagccgcgcctttttcccggcctgctacagcggcgacgggcgcctgaagtcctgggggctgacgctgcgctgcgcgctcgtcgtcttcacgctgctcatggccatttatgtgccgcacttcgcgctgctcatgggcctcaccggcagcctcacgggcgccggcctctgtttcttgctgcccagcctctttcacctgcgcctgctctggcgcaagctgctgtggcaccaagtcttcttcgacgtcgccatcttcgtcatcggcggcatctgcagcgtgtccggcttcgtgcactccctcgagggcctcatcgaagcctaccgaaccaacgcggaggactagggcgcaagggcgagcccccgccgcgcttctgcgctctctcccttctcccctcaccccgcccccaccagcccagtgcgccctgccgccgcgcttgggaggccaagctttaaacatctctggttcctagtttctgattattcggggatgggggggatgggaggggacagggattcacgatccatcgcgtctgcgtttctgttgtcctttcttttccacaacaccctggttttggggggaggcggggtgcatttgcgggcagggttctctgtccttccaagtggggccccgacactttggttccagtcatcgagggggttgggaagggagggagagggggcgcagctcgcaggcgtggcaacttgaccttgggggaatatttcacatccatccagagctcggaatctacagcgtccagccatttccagcaagagcgcttcccattccggagacgtttcaaccctgcagcgggaaaggctgactgggaaatccattttgggtgggcaatttccttcaacgaagccggaaggcgagaagccgcggcggggccagcttgcctgccggttttcaggaatctaaactctcatcttgtgcaatttatcaggtgtggaactgttctactgtgcgtgtggtgtgctcgtggtgaataagatgaaatgtatatcagaaaaaaatctatctctaatttagagtgcggtacataattatatccgcaaataaagaagagacaaaggcttgaaaaaaaaaaaaaaaaaaaaaaaaa

SEQ ID NO: 10 - protein, 525 aa, Homo sapiens
MATLLRSKLSNVATSVSNKSQAKMSGMFARMGFQAATDEEAVGFAHCDDLDFEHRQGLQMDILKAEGEPCGDEGAEAPVEGDIHYQRGSGAPLPPSGSKDQVGGGGEFGGHDKPKITAWEAGWNVTNAIQGMFVLGLPYAILHGGYLGLFLIIFAAVVCCYTGKILIACLYEENEDGEVVRVRDSYVAIANACCAPRFPTLGGRVVNVAQIIELVMTCILYVVVSGNLMYNSFPGLPVSQKSWSIIATAVLLPCAFLKNLKAVSKFSLLCTLAHFVINILVIAYCLSRARDWAWEKVKFYIDVKKFPISIGIIVFSYTSQIFLPSLEGNMQQPSEFHCMMNWTHIAACVLKGLFALVAYLTWADETKEVITDNLPGSIRAVVNIFLVAKALLSYPLPFFAAVEVLEKSLFQEGSRAFFPACYSGDGRLKSWGLTLRCALVVFTLLMAIYVPHFALLMGLTGSLTGAGLCFLLPSLFHLRLLWRKLLWHQVFFDVAIFVIGGICSVSGFVHSLEGLIEAYRTNAED

SEQ ID NO: 11 - DNA, 1062 bp, Homo sapiens
  CDS (1)...(1062)
atggaaaccaacttctccattcctctgaatgaaactgaggaggtgctccctgagcctgctggccacaccgttctgtggatcttctcattgctagtccacggagtcacctttgtcttcggggtcctgggcaatgggcttgtgatctgggtggctggattccggatgacacgcacagtcaacaccatctgttacctgaacctggccctagctgacttctctttcagtgccatcctaccattccgaatggtctcagtcgccatgagagaaaaatggccttttgcgtcattcctatgtaagttagttcatgttatgatagacatcaacctgtttgtcagtgtctacctgatcaccatcattgctctggaccgctgtatttgtgtcctgcatccagcctgggcccagaaccatcgcaccatgagtctggccaagagggtgatgacgggactctggattttcaccatagtccttaccttaccaaatttcatcttctggactacaataagtactacgaatggggacacatactgtattttcaactttgcattctggggtgacactgctgtagagaggttgaacgtgttcattaccatggccaaggtctttctgatcctccacttcattattggcttcacggtgcctatgtccatcatcacagtctgctatgggatcatcgctgccaaaattcacagaaaccacatgattaaatccagccgtcccttacgtgtcttcgctgctgtggtggcttctttcttcatctgttggttcccttatgaactaattggcattctaatggcagtctggctcaaagagatgttgttaaatggcaaatacaaaatcattcttgtcctgattaacccaacaagctccttggccttttttaacagctgcctcaacccaattctctacgtctttatgggtcgtaacttccaagaaagactgattcgctctttgcccactagtttggagagggccctgactgaggtccctgactcagcccagaccagcaacacacacaccacttctgcttcacctcctgaggagacggagttacaagcaatgtga

SEQ ID NO: 12 - protein, 353 aa, Homo sapiens
METNFSIPLNETEEVLPEPAGHTVLWIFSLLVHGVTFVFGVLGNGLVIWVAGFRMTRTVNTICYLNLALADFSFSAILPFRMVSVAMREKWPFASFLCKLVHVMIDINLFVSVYLITIIALDRCICVLHPAWAQNHRTMSLAKRVMTGLWIFTIVLTLPNFIFWTTISTTNGDTYCIFNFAFWGDTAVERLNVFITMAKVFLILHFIIGFTVPMSIITVCYGIIAAKIHRNHMIKSSRPLRVFAAVVASFFICWFPYELIGILMAVWLKEMLLNGKYKIILVLINPTSSLAFFNSCLNPILYVFMGRNFQERLIRSLPTSLERALTEVPDSAQTSNTHTTSASPPEETELQAM

SEQ ID NO: 13 - DNA, 4221 bp, Homo sapiens
  CDS (51)...(2957)
cagcccgagcccgagcccgagcccgagccggcgccaccgcgcccccggccatggcttttgccaatttccgccgcatcctgcgcctgtctaccttcgagaagagaaagtcccgcgaatatgagcacgtccgccgcgacctggaccccaacgaggtgtgggagatcgtgggcgagctgggcgacggcgccttcggcaaggtttacaaggccaagaataaggagacgggtgctttggctgcggccaaagtcattgaaaccaagagtgaggaggagctggaggactacatcgtggagattgagatcctggccacctgcgaccacccctacattgtgaagctcctgggagcctactatcacgacgggaagctgtggatcatgattgagttctgtccagggggagccgtggacgccatcatgctggagctggacagaggcctcacggagccccagatacaggtggtttgccgccagatgctagaagccctcaacttcctgcacagcaagaggatcatccaccgagatctgaaagctggcaacgtgctgatgaccctcgagggagacatcaggctggctgactttggtgtgtctgccaagaatctgaagactctacagaaacgagattccttcatcggcacgccttactggatggcccccgaggtggtcatgtgtgagaccatgaaagacacgccctacgactacaaagccgacatctggtccctgggcatcacgctgattgagatggcccagatcgagccgccacaccacgagctcaaccccatgcgggtcctgctaaagatcgccaagtcagaccctcccacgctgctcacgccctccaagtggtctgtagagttccgtgacttcctgaagatagccctggataagaacccagaaacccgacccagtgccgcgcagctgctggagcatcccttcgtcagcagcatcaccagtaacaaggctctgcgggagctggtggctgaggccaaggccgaggtgatggaagagatcgaagacggccgggatgagggggaagaggaggacgccgtggatgccgcctccaccctggagaaccatactcagaactcctctgaggtgagtccgccaagcctcaatgctgacaagcctctcgaggagtcaccttccaccccgctggcacccagccagtctcaggacagtgtgaatgagccctgcagccagccctctggggacagatccctccaaaccaccagtcccccagtcgtggcccctggaaatgagaacggcctggcagtgcctgtgcccctgcggaagtcccgacccgtgtcaatggatgccagaattcaggtagcccaggagaagcaagttgctgagcagggtggggacctcagcccagcagccaacagatctcaaaaggccagccagagccggcccaacagcagcgccctggagaccttgggtggggagaagctggccaatggcagcctggagccacctgcccaggcagctccagggccttccaagagggactcggactgcagcagcctctgcacctctgagagcatggactatggtaccaatctctccactgacctgtcgctgaacaaagagatgggctctctgtccatcaaggacccgaaactgtacaaaaaaaccctcaagcggacacgcaaatttgtggtggatggtgtggaggtgagcatcaccacctccaagatcatcagcgaagatgagaagaaggatgaggagatgagatttctcaggcgccaggaactccgagagcttcggctgctccagaaagaagagcatcggaaccagacccagctgagtaacaagcatgagctgcagctggagcaaatgcataaacgttttgaacaggaaatcaacgccaagaagaagttctttgacacggaattagagaacctggagcgtcagcaaaagcagcaagtggagaagatggagcaagaccatgccgtgcgccgccgggaggaggccaggcggatccgcctggagcaggatcgggactacaccaggttccaagagcagctcaaactgatgaagaaagaggtgaagaacgaggtggagaagctcccccgacagcagcggaaggaaagcatgaagcagaagatggaggagcacacgcagaaaaagcagcttcttgaccgggactttgtagccaagcagaaggaggacctggagctggccatgaagaggctcaccaccgacaacaggcgggagatctgtgacaaggagcgcgagtgcctcatgaagaagcaggagctccttcgagaccgggaagcagccctgtgggagatggaagagcaccagctgcaggagaggcaccagctggtgaagcagcagctcaaagaccagtacttcctccagcggcacgagctgctgcgcaagcatgagaaggagcgggagcagatgcagcgctacaaccagcgcatgatagagcagctgaaggtgcggcagcaacaggaaaaggcgcggctgcccaagatccagaggagtgagggcaagacgcgcatggccatgtacaagaagagcctccacatcaacggcgggggcagcgcagctgagcagcgtgagaagatcaagcagttctcccagcaggaggagaagaggcagaagtcggagcggctgcagcaacagcagaaacacgagaaccagatgcgggacatgctggcgcagtgtgagagcaacatgagcgagctgcagcagctgcagaatgaaaagtgccacctcctggtagagcacgaaacccagaaactgaaggccctggatgagagccataaccagaacctgaaggaatggcgggacaagcttcggccgcgcaagaaggctctggaagaggatctgaaccagaagaagcgggagcaggagatgttcttcaagctgagcgaggaggcggagtgcccaaacccctccaccccaagcaaggccgccaagttcttcccctacagttctgcggatgcttcttaacaaccgcccggggctgtggctggcagcttggtgggccccagggccttctccttcattctctgtgaacatgtaactcaggaccccttttccctcttgcgtctgtgccagctcaaatccagcccctcgccctgtgccaccccaactgtgcctgatagacctgccccagcgttcctgacttcttgctggcctgtggagggtgaggtgtaattatttgtcacctgaacctaatgtatattctccttgagccccagatcccttcaagctggaagggatggggctgttggtggggtcagggtccaagaggaatgggtgttctgtggcctcgagtcctctcctgtttgcgaaaataccagttttgctctctgtgggacaaagcactgctgatgaagtccccgtgggctcatccgggctggaattcttggtttttcagccattccctgcagagtcactcaatcatcaagtccctcacagccatttctgttcccagaggagccaggcctgcagctggctgctcaggagatggcctcattcctcctgttctcccagtttgctttccacttaagacaaagccttgtctatgtggggggcggggaccggggaaagagggaggctgaaatgtttattctgcttctcccgtgtttcatgccatctcgcgtcccccttcctgcacatgggtgtgaatgcacacacatacgcgtacacacaggacttggtctgctggcctggcctcttctgcccaggtgggttggaacacgtttgctgcctgagcctgtgccactgagcatgttaggtggagcagttggtgtggcacgtgcggggtgttggcaccggaggcatggaaaagcacaggctgtactgccaggctgcgatgcgtgctggcccccgcacaggctcctgtgtgcagggactgattcctcagcacacgaggcttccacaacccagtctgctccatagcactctggcccaccctgtctgcaggtgaaacaggagggctgtttgcctctgccccatccccccgactgtgttcaggagtcccaccttgcatttcagacctgggctggcagtctgttggacttctcttcaggaagaaaaagcatcagggggaaatggaatgcccctgccccaggaacatggcagaagcacaggttctgtacctcagatggactcctgctgggccttcggggtctcagttggcttcccccagattctgattctacagctgcagaatgtatataacacaataaaagcaaatgtttgaaccagt

SEQ ID NO: 14 - protein, 968 aa, Homo sapiens
MAFANFRRILRLSTFEKRKSREYEHVRRDLDPNEVWEIVGELGDGAFGKVYKAKNKETGALAAAKVIETKSEEELEDYIVEIEILATCDHPYIVKLLGAYYHDGKLWIMIEFCPGGAVDAIMLELDRGLTEPQIQVVCRQMLEALNFLHSKRIIHRDLKAGNVLMTLEGDIRLADFGVSAKNLKTLQKRDSFIGTPYWMAPEVVMCETMKDTPYDYKADIWSLGITLIEMAQIEPPHHELNPMRVLLKIAKSDPPTLLTPSKWSVEFRDFLKIALDKNPETRPSAAQLLEHPFVSSITSNKALRELVAEAKAEVMEEIEDGRDEGEEEDAVDAASTLENHTQNSSEVSPPSLNADKPLEESPSTPLAPSQSQDSVNEPCSQPSGDRSLQTTSPPVVAPGNENGLAVPVPLRKSRPVSMDARIQVAQEKQVAEQGGDLSPAANRSQKASQSRPNSSALETLGGEKLANGSLEPPAQAAPGPSKRDSDCSSLCTSESMDYGTNLSTDLSLNKEMGSLSIKDPKLYKKTLKRTRKFVVDGVEVSITTSKIISEDEKKDEEMRFLRRQELRELRLLQKEEHRNQTQLSNKHELQLEQMHKRFEQEINAKKKFFDTELENLERQQKQQVEKMEQDHAVRRREEARRIRLEQDRDYTRFQEQLKLMKKEVKNEVEKLPRQQRKESMKQKMEEHTQKKQLLDRDFVAKQKEDLELAMKRLTTDNRREICDKERECLMKKQELLRDREAALWEMEEHQLQERHQLVKQQLKDQYFLQRHELLRKHEKEREQMQRYNQRMIEQLKVRQQQEKARLPKIQRSEGKTRMAMYKKSLHINGGGSAAEQREKIKQFSQQEEKRQKSERLQQQQKHENQMRDMLAQCESNMSELQQLQNEKCHLLVEHETQKLKALDESHNQNLKEWRDKLRPRKKALEEDLNQKKREQEMFFKLSEEAECPNPSTPSKAAKFFPYSSADAS

SEQ ID NO: 15 - DNA, 3513 bp, Homo sapiens
  CDS (82)...(3150)
cgcacccagtcaccagcgttcgggagcctgtcgcagcgggaccgacggaatccggagcaggcgacagggcgcagaagcgggatgtacttctgttggggcgccgactccagggagctgcagcgccggaggacggcgggcagccccggggctgagctactgcaggcggccagcggggagcgccactctctgctgctgctgaccaaccacagggtcctctcgtgcggagacaacagcaggggtcagctgggccgcaggggcgcgcagcgcggggagctgccagaaccaattcaggcattggaaaccctaattgttgatctcgtgagctgcgggaaggagcactccctggctgtgtgccacaaaggaagggtcttcgcatggggagctggttctgaagggcagctggggattggagaattcaaggaaataagtttcacacctaagaaaataatgactctgaatgatataaaaataatacaagtttcctgtggacactaccactccctggcattatcaaaagatagccaagtgttttcgtggggaaagaacagccatgggcagctgggcttggggaaggagttcccctcccaagccagcccgcagagggtgaggtccctggaggggatcccactggctcaggtggctgccggaggggctcacagctttgccctgtctctctgtgggacttcgtttggctggggaagtaacagtgccgggcagctggccctcagtgggcgtaatgtcccagtgcaaagcaacaagcctctctcagtcggtgcactgaagaatctaggtgtggtttatatcagctgtggtgatgcacacactgcggtgcttacccaggacgggaaagtgttcacatttggagacaatcgctctggacagctgggatacagccccactcctgagaagagaggtccacaacttgtggaaagaattgatggcctagtttcgcagatagattgtggaagttatcacaccctggcatatgtgcacaccactggtcaggtggtatcttttggtcatggaccaagtgacacaagcaagccaactcatccggaggccctgacagagaactttgacattagctgcctgatttctgctgaagacttcgtggatgttcaagtcaaacacatttttgctggaacatatgccaactttgtgacaactcatcaggatactagttccacacgtgctcccgggaaaaccctgccagaaataagccgaattagccagtccatggcagaaaaatggatagcagtgaaaagaagaagtactgaacatgaaatggctaaaagtgaaattagaatgatattttcatctcctgcttgtctgactgcaagttttttaaagaaaagaggaactggagaaacgacttccattgatgtggacttagaaatggcaagagataccttcaagaagttaacaaaaaaggaatggatttcttccatgataactacgtgtctcgaggatgatctgctcagagctcttccatgccattctccacaccaagaagctttatcagttttcctcctgctcccagaatgtcctgtgatgcatgattctaagaactggaagaacctggtggttccatttgcaaaggctgtgtgtgaaatgagtaaacaatctttgcaagtcctaaagaagtgttgggcatttttgcaagaatcttctctgaatccgctgatccagatgcttaaagcagccatcatctctcagctgcttcatcagactaaaaccgaacaggatcactgtaatgttaaagctcttttaggaatgatgaaagaactgcataaggtaaacaaagctaactgtcgactaccagaaaatactttcaacataaatgaactctccaacttattaaacttttatatagatagaggaagacagctctttcgggataaccacctgatacctgcagaaacccccagtcctgttattttcagtgattttccatttatctttaattcgctatccaaaattaaattattgcaagctgattcacatataaagatgcagatgtcagaaaagaaagcatacatgcttatgcatgaaacaattctgcaaaaaaaggatgaatttcctccatcacccagatttatacttagagtcagacgaagtcgcctggttaaagatgctctgcgtcaattaagtcaagctgaagctactgacttctgcaaagtattagtggttgaatttattaatgaaatttgtcctgagtctggaggggttagttcagagttcttccactgtatgtttgaagagatgaccaagccagaatatggaatgttcatgtatcctgaaatgggttcctgcatgtggtttcctgccaagcctaaacctgagaagaaaagatatttcctctttggaatgctgtgtggactctccttattcaatttaaatgttgctaaccttcctttcccactggctctgtataaaaaacttctggaccaaaagccatcattggaagatttaaaagaactcagtcctcggttggggaagagtttgcaagaagttctagatgatgctgctgatgacattggagatgcgctctgcatacgcttttctatacactgggaccaaaatgatgttgacttaattccaaatgggatctccatacctgtggaccaaaccaacaagagagactatgtttctaagtatattgattacattttcaacgtctctgtaaaagcagtttatgaggaatttcagagaggattttatagagtctgtgagaaggagatacttagacatttctaccctgaagaactaatgacagcaatcattggaaatactgattatgactggaaacagtttgaacagaattcaaagtatgagcaaggataccaaaaatcacatcctactatacagttgttttggaaggctttccacaaactaaccttggatgaaaagaaaaaattcctctttttccttacaggacgtgataggctgcatgcaagaggcatacagaaaatggaaatagtatttcgctgtcctgaaactttcagtgaaagagatcacccaacatcaataacttgtcataatattctctccctccctaagtattctacaatggaaagaatggaggaagcacttcaagtagccatcaacaacaacagaggatttgtctcacccatgctcacacagtcataatcacctctgagagactcagggtgggctttctcacacttggatccttctgttcttccttacacctaaataatacaagagattaatgaatagtggttagaagtagttgagggagagattgggggaatggggagatgatgatgatggtcaaagggtgcaaaatctcacacaagactgaggcaggagaatagggtacagagatagggatctaaggatgacttggacacactccctggcactgaagagtctgaacactggcctgtgattggtccattccaggaccttcatttgcataaggtatcaaaccacatcagcctctgattggccatgggccagacctgcactctggccaatgattggttcatt

SEQ ID NO: 16 - protein, 1022 aa, Homo sapiens
MYFCWGADSRELQRRRTAGSPGAELLQAASGERHSLLLLTNHRVLSCGDNSRGQLGRRGAQRGELPEPIQALETLIVDLVSCGKEHSLAVCHKGRVFAWGAGSEGQLGIGEFKEISFTPKKIMTLNDIKIIQVSCGHYHSLALSKDSQVFSWGKNSHGQLGLGKEFPSQASPQRVRSLEGIPLAQVAAGGAHSFALSLCGTSFGWGSNSAGQLALSGRNVPVQSNKPLSVGALKNLGVVYISCGDAHTAVLTQDGKVFTFGDNRSGQLGYSPTPEKRGPQLVERIDGLVSQIDCGSYHTLAYVHTTGQVVSFGHGPSDTSKPTHPEALTENFDISCLISAEDFVDVQVKHIFAGTYANFVTTHQDTSSTRAPGKTLPEISRISQSMAEKWIAVKRRSTEHEMAKSEIRMIFSSPACLTASFLKKRGTGETTSIDVDLEMARDTFKKLTKKEWISSMITTCLEDDLLRALPCHSPHQEALSVFLLLPECPVMHDSKNWKNLVVPFAKAVCEMSKQSLQVLKKCWAFLQESSLNPLIQMLKAAIISQLLHQTKTEQDHCNVKALLGMMKELHKVNKANCRLPENTFNINELSNLLNFYIDRGRQLFRDNHLIPAETPSPVIFSDFPFIFNSLSKIKLLQADSHIKMQMSEKKAYMLMHETILQKKDEFPPSPRFILRVRRSRLVKDALRQLSQAEATDFCKVLVVEFINEICPESGGVSSEFFHCMFEEMTKPEYGMFMYPEMGSCMWFPAKPKPEKKRYFLFGMLCGLSLFNLNVANLPFPLALYKKLLDQKPSLEDLKELSPRLGKSLQEVLDDAADDIGDALCIRFSIHWDQNDVDLIPNGISIPVDQTNKRDYVSKYIDYIFNVSVKAVYEEFQRGFYRVCEKEILRHFYPEELMTAIIGNTDYDWKQFEQNSKYEQGYQKSHPTIQLFWKAFHKLTLDEKKKFLFFLTGRDRLHARGIQKMEIVFRCPETFSERDHPTSITCHNILSLPKYSTMERMEEALQVAINNNRGFVSPMLTQS

SEQ ID NO: 17 - DNA, 3923 bp, Homo sapiens
  CDS (230)...(1954)
gagccagcgagccagcgcgcgcgggcgggcggacagatcggagccgagcggggccgggcggggcgctccctgcagggctctgcgcggcgtgccgcggcggccgcgggctccggccccgggccatgagcccctccgcgactcggcgctgagcccgccaccggtccagcgccccaggacccgccgccggctgccggcttgccgaagccccctcaggatcccctcaacaaggatggaactgaaggccgaggaggaggaggtgggtggcgtccagccggtgagcatccaggccttcgccagcagctccacactgcacggcctggcccacatcttctcctacgagcggctgtctctgaagcgggcactgtgggccctgtgcttcctgggctcgctggctgtgctgctgtgtgtgtgcacggagcgtgtgcagtactacttccactaccaccatgtcaccaagctcgacgaggtggctgcctctcagcttaccttccctgctgtcacgctgtgcaacctcaacgagttccgctttagccaagtctccaagaatgacctgtatcatgctggggagctgctggccctgctcaacaacaggtatgagataccagacacacagatggcagatgaaaagcagctggagatactgcaggacaaagccaacttccgcagcttcaaacccaaacccttcaacatgcgtgagttctacgaccgagctgggcacgacattcgagacatgctgctctcctgccacttccggggggaggtctgcagcgctgaagacttcaaggtggtcttcacacgctatggaaagtgctacacgttcaactcgggccgagatgggcggccgcggctgaagaccatgaaggatgggacgggcaatgggctggaaatcatgctggacatccagcaggacgagtacctgcctgtgtggggggagactgacgagacgtccttcgaagcaggcatcaaagtgcagatccatagtcaggatgaacctcctttcatcgaccagctgggctttggcgtggccccaggcttccagacctttgtggcctgccaggagcagcggctcatctacctgcccccaccctggggcacctgcaaagctgttaccatggactcggatttggatttcttcgactcctacagcatcactgcctgccgcatcgactgtgagacgcgctacctggtggagaactgcaactgccgcatggtgcacatgccaggggatgccccatactgtactccagagcagtacaaggagtgtgcagatcctgctctggacttcctggtggagaaggaccaggagtactgcgtgtgtgaaatgccttgcaacctgacccgctatggcaaagagctgtccatggtcaagatccccagcaaagcctcagccaagtacctggccaagaagttcaacaaatctgagcaatacataggggagaacatcctggtgctggacattttctttgaagtcctcaactatgagaccattgaacagaagaaggcctatgagattgcagggctcctgggtgagctgctgatgacacctgtccccttctcatgccatgggcatggcgtggctccctatcatccaaaagcagggtgctcacttctgtcccatgagggtcctccaccccagaggcccttccccaaaccctgttgtcttggtgacatcgggggccagatggggctgttcatcggggccagcatcctcacggtgctggagctctttgactacgcctacgaggtcattaagcacaagctgtgccgacgaggaaaatgccagaaggaggccaaaaggagcagtgcggacaagggcgtggccctcagcctggacgacgtcaaaagacacaacccgtgcgagagccttcggggccaccctgccgggatgacatacgctgccaacatcctacctcaccatccggcccgaggcacgttcgaggactttacctgctgagccccgcaggccgctgaaccaaaggcctagatggggaggactaggagagcgrgggggcccccagctgcctcctcacatctgccctgggractccccacactccggggcagatctttcctcttgtctgtggtaaggaaggagtcttgaccatagagtcctctctctgcctctatcccattcyttttacatttaacaaaactaatctaaaaaagaactaaaaagggagaacggggcaagggacctcaggctgcccctctctcctccatgctgcctcccctagctcccagcctgaattctgtctatctagctgtctgccatctgagtgtccatctacattctgctgccaccagtcaccaaaggcccttcccagtgaggggtggaagggatctctggggtctggaatttggccccaaaccagagaatgtaccttaagggggagggctagtgtgggggagggaggcttccccagccttaagagaccctctcagcccagtgactgtccccaaacccaagtctcctggcaggaactaaaacctcagccccactctctcacaccatgtggaatctcgtgggggtcggggatccccttaagaagtggtaatggggacaagatgcggccctggtgctgtaggctacatcctgatacctataagttcacccccaccccacagctgctggagagaaatcccaagaggcagcccttcctcaccatcccattaaagacckggctggttagcgtccagctcagggagaagggcgctagtgcctaacctcactggtccctctcccggaggcccttgtagagggccacgtccataaattttcttatggaactctcccacatcctcttccccaacttcatttgcttctctcaacaacctcatctgcattttctatttctatatgatacagactctatattgctatatctctgtatatactttcccagccctgtctgtctccaccccatcccctcttgtctctgagaaccattctcccaccccaagttccaccttctatgtttctactccctccctggtctctgaatgccttygcctgtataaagagttggactctctcccctggtgtctgtactgtgtacacacatccctctgagaagcacaaggagacgacacgcgcattgtaacctttgcactgtctcagtggcgacaaaggaagctgtgaatcacaagctctgcctctttctggcctcaccctctcccccaacccgggcaccctcggccctccctgcagccttaacattctcttcccctgctcctcctatcccattgccctctgcccagctgacagtggcatccccagggaaggggttgctgtagagatagcccccacccaggggatggaggtctaccctggacactaagccaagtgtgtcagagacagaagggagctggggattggcgactcctgaagttggggcagtgggatgctgacaggcagaagctgaggtcctcagtcagtggcctttcctccttctgggtgcccagccccctttcctcacctgatacccaagcccaccacttttattttctggtgaggtgggtttgggaggaaagagaggcctagaggaggagttgaaagctctgctgttgtctcaccctatcttaatgagagacaagtgaggtggagggcctgccccccctccctccaccagacactccttccaggcctgagccccaacccctcttcaggccttccttccctagctgtgtcttggtcttcaatcccagaacaggacctgtgagcagctgcattggcctggagctggagagtaaggctgtaggatctttggaatctcttggttcctaagagtttcctcagagatcatacctccccagagggaagcaggaatgaggccaaaaagtgtgcattggataggggaacagcaggcagggctctgggtgacgcatgcctctggtctaataaactgggtttcaaccaaaaaaaa

SEQ ID NO: 18 - protein, 574 aa, Homo sapiens [sequence omitted]

SEQ ID NO: 19 - DNA, 1373 bp, Homo sapiens
  CDS (31)...(1373)
gaattysggktcrmmcmcgsstccscagcaatgtctccaccactgctgctgctacccttgctgctgctgctgcctctgctgaatgtggagcctgctggggccacactgatccggatccctcttcgtcaagtccaccctggacgcaggaccctgaacctactgaggggatggggaaaaccagcagagctccccaagttgggggccccatcccctggggacaagcctgcctcggtacctctctccaaattcctggatgcccagtattttggggaaattgggctgggaacgcctccacaaaacttcactgttgcctttgacactggctcctccaatctctgggtcccgtccaggagatgccacttcttcagtgtgccctgctggttccaccaccgcttcaatcccaatgcctccagctccttcaagcccagtgggaccaagtttgccattcagtatggaactgggcgggtagatggaatcctgagtgaggacaagctgactattggtggaatcaagggtgcatccgtgattttcggggaagctctgtgggaatccagcctggtcttcactgtttcccgccccgatgggatattgggcctcggttttcccattctgtctgtggaaggagttcggcccccgctggatgtactggtggagcaggggctattggataagcctgtcttctccttttacttcaacagggaccctgaagtggctgatggaggagagctggtcctggggggctcagacccggcacactacatcccacccctcaccttcgtgccagtcacagtccccgcctactggcagatccacatggagcgtgtgaaggtgggctcacggctgactctctgtgcccagggctgtgctgccatcctggatacaggcacacctgtcatcgtaggacccactgaggagatccgggccctgcatgcagccattgggggaatccccttgctggctggggagtacatcatccggtgctcagaaatcccaaagctccccgcagtctcactcctcattgggggggtctggtttaatctcacggcccaggattacgtcatccagtttgctcagggtgacgtccgcctctgcttgtccggcttccgggccttggacatcgcttcgcctccagtacctgtgtggatcctcggcgacgttttcttgggggcgtatgtgaccgtcttcgaccgcggggacatgaagagcggcgcacgagtgggactggcgcgcgctcgccctcgcggagcggacctgggaaggcgcgagaccgcgcaggcgcagtaccgcgggtgccgcccaggtgatgcgcatgcgcaccgggtagcagagctagcgctactcagtaaaaatccaatatttccattgaaaaaaaa

SEQ ID NO: 20 - protein, 448 aa, Homo sapiens
  VARIANT (448)...(448): Xaa = Any Amino Acid
MSPPLLLLPLLLLLPLLNVEPAGATLIRIPLRQVHPGRRTLNLLRGWGKPAELPKLGAPSPGDKPASVPLSKFLDAQYFGEIGLGTPPQNFTVAFDTGSSNLWVPSRRCHFFSVPCWFHHRFNPNASSSFKPSGTKFAIQYGTGRVDGILSEDKLTIGGIKGASVIFGEALWESSLVFTVSRPDGILGLGFPILSVEGVRPPLDVLVEQGLLDKPVFSFYFNRDPEVADGGELVLGGSDPAHYIPPLTFVPVTVPAYWQIHMERVKVGSRLTLCAQGCAAILDTGTPVIVGPTEEIRALHAAIGGIPLLAGEYIIRCSEIPKLPAVSLLIGGVWFNLTAQDYVIQFAQGDVRLCLSGFRALDIASPPVPVWILGDVFLGAYVTVFDRGDMKSGARVGLARARPRGADLGRRETAQAQYRGCRPGDAHAHRVAELALLSKNPIFPLKKX

SEQ ID NO: 21 - DNA, 1506 bp, Homo sapiens
  CDS (1)...(1506)
atgtcatcctcaggcacgccagacttacctgtcctactcaccgatttgaagattcaatatactaagatcttcataaacaatgaatggcatgattcagtgagtggcaagaaatttcctgtctttaatcctgcaactgaggaggagctctgccaggtagaagaaggagataaggaggatgttgacaaggcagtgaaggccgcaagacaggcttttcagattggatctccgtggcgtactatggatgcttccgagagggggcgactattatacaagttggctgatttaatcgaaagagatcgtctgctgctggcgacaatggagtcaatgaatggtggaaaactctattccaatgcatatctgagtgatttagcaggctgcatcaaaacattgcgctactgtgcaggttgggctgacaagatccagggccgtacaataccaattgatggaaatttttttacatatacaagacatgaacctattggtgtatgtggccaaatcattccttggaatttcccgttggttatgctcatttggaagatagggcctgcactgagctgtggaaacacagtggttgtcaaaccagcagagcaaactcctctcactgctctccacgtggcatctttaataaaagaggcagggtttcctcctggagtagtgaatattgttcctggttatgggcctacagcaggggcagccatttcttctcacatggatatagacaaagtagccttcacaggatcaacagaggttggcaagttgatcaaagaagctgccgggaaaagcaatctgaagagggtgaccctggagcttggaggaaagagcccttgcattgtgttagctgatgccgacttggacaatgctgttgaatttgcacaccatggggtattctaccaccagggccagtgttgtatagccgcatccaggatttttgtggaagaatcaatttatgatgagtttgttcgaaggagtgttgagcgggctaagaagtatatccttggaaatcctctgaccccaggagtcactcaaggccctcagattgacaaggaacaatatgataaaatacttgacctcattgagagtgggaagaaagaaggggccaaactggaatgtggaggaggcccgtgggggaataaaggctactttgtccagcccacagtgttctctaatgttacagatgagatgcgcattgccaaagaggagatttttggaccagtgcagcaaatcatgaagtttaaatctttagatgacgtgatcaaaagagcaaacaatactttctatggcttatcagcaggagtgtttaccaaagacattgataaagccataacaatctcctctgctctgcaggcaggaacagtgtgggtgaattgctatggcgtggtaagtgcccagtgcccctttggcggattcaagatgtctggaaatggaagagaactgggagagtacggtttccatgaatatacagaggtcaaaacagtcacagtgaaaatctctcagaagaactcataa

SEQ ID NO: 22 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLSDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 23 - DNA, 3257 bp, Homo sapiens
  CDS (136)...(2199)
aacaggcgtgacgccagttctaaacttgaaacaaaacaaaacttcaaagtacaccaaaatagaacctccttaaagcataaatctcacggagggtctcggccgccagtggaaggagccaccgcccccgccccgaccatggccgaggagctggtcttagagaggtgtgatctggagctggagaccaatggccgagaccaccacacggccgacctgtgccgggagaagctggtggtgcgacggggccagcccttctggctgaccctgcactttgagggccgcaactaccaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttcaagatgggatcctagacatctgcctgatccttctagatgtcaaccccaagttcctgaagaacgccggccgtgactgctcccggcgcagcagccccgtctacgtgggccgggtgggtagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctaggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaaggaacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctaaccctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctcgtgccgctccacatgggcctccacaagctggtggtgaacttcgagagcgacaagctgaaggctgtgaagggcttccggaatgtcatcattggccccgcctaagggacccctgctcccagcctgctgagagcccccaccttgatcccaatccttatcccaagctagtgagcaaaatatgccccttattgggccccagaccccagggcagggtgggcagcctatgggggctctcggaaatggaatgtgcccctggcccatctcagcctcctgagcctgtgggtccccactcaccccctttgctgtgaggaatgctctgtgccagaaacagtgggagccctgacctgtgctgactggggctggggtgagagaggaaagacctacattccctctcctgcccagatgccctttggaaagccattgaccacccaccatattgtttgatctacttcatagctccttggagcaggcaaaaaagggacagcatgcccttggctggatcaggaatccagctccctagactgcatcccgtacctcttcccatgactgcacccagctccaggggcccttgggacacccagagctgggtggggacagtgataggcccaaggtcccctccacatcccagcagcccaagcttaatagccctccccctcaacctcaccattgtgaagcacctactatgtgctgggtgcctcccacacttgctggggctcacggggcctccaacccatttaatcaccatgggaaactgttgtgggcgctgcttccaggataaggagactgaggcttagagagaggaggcagccccctccacaccagtggcctcgtggttataagcaaggctgggtaatgtgaaggcccaagagcagagtctgggcctctgactctgagtccactgctccatttataaccccagcctgacctgagactgtcgcagaggctgtctggggcctttatcaaaaaaagactcagccaagacaaggaggtagagaggggactgggggactgggagtcagagccctggctgggttcaggtcccacgtctggccagcgactgccttctcctctctgggcctttgtttccttgttggtcagaggagtgattgaacctgctcatctccaaggatcctctccactccatgtttgcaatacacaattcc

SEQ ID NO: 24 - protein, 687 aa, Homo sapiens
MAEELVLERCDLELETNGRDHHTADLCREKLVVRRGQPFWLTLHFEGRNYQASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFQDGILDICLILLDVNPKFLKNAGRDCSRRSSPVYVGRVGSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLTLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLVPLHMGLHKLVVNFESDKLKAVKGFRNVIIGPA

SEQ ID NO: 25 - DNA, 1061 bp, Homo sapiens
  CDS (11)...(1051)
ggccaccaccatggatacaggccccgaccagtcctacttctccggcaatcactggttcgtcttctcggtgtaccttctcactttcctggtggggctccccctcaacctgctggccctggtggtcttcgtgggcaagctgcagcgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttccggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctgttggcggccacgctgctcaacttccttgtctgctttgggccctacaacgtgtcccatgtcgtgggctatatctgcggtgaaagcccggcatggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaagctaggtcctccggg

SEQ ID NO: 26 - protein, 346 aa, Homo sapiens
MDTGPDQSYFSGNHWFVFSVYLLTFLVGLPLNLLALVVFVGKLQRRPVAVDVLLLNLTASDLLLLLFLPFRMVEAANGMHWPLPFILCPLSGFIFFTTIYLTALFLAAVSIERFLSVAHPLWYKTRPRLGQAGLVSVACWLLASAHCSVVYVIEFSGDISHSQGTNGTCYLEFRKDQLAILLPVRLEMAVVLFVVPLIITSYCYSRLVWILGRGGSHRRQRRVAGLLAATLLNFLVCFGPYNVSHVVGYICGESPAWRIYVTLLSTLNSCVDPFVYYFSSSGFQADFHELLRRLCGLWGQWQQESSMELKEQKGGEEQRADRPAERKTSEHSQGCGTGGQVACAES

SEQ ID NO: 27 - DNA, 1013 bp, Homo sapiens
  CDS (11)...(1003)
cccctccaggatgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccttgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtagcagtttccct

SEQ ID NO: 28 - protein, 330 aa, Homo sapiens
MLPDWKSSLILMAYIIIFLTGLPANLLALRAFVGRIRQPQPAPVHILLLSLTLADLLLLLLLPFKIIEAASNFRWYLPKVVCALTSFGFYSSIYCSTWLLAGISIERYLGVAFPVQYKLSRRPLYGVIAALVAWVMSFGHCTIVIIVQYLNTTEQVRSGNEITCYENFTDNQLDVVLPVRLELCLVLFFIPMAVTIFCYWRFVWIMLSQPLVGAQRRRRAVGLAVVTLLNFLVCFGPYNVSHLVGYHQRKSPWWRSIAVVFSSLNASLDPLLFYFSSSVVRRAFGRGLQVLRNQGSSLLGRRGKDTAEGTNEDRGVGQGEGMPSSDFTTE

SEQ ID NO: 29 - DNA, 1982 bp, Homo sapiens
  CDS (63)...(1460)
ggcgagaggcgggctgaggcggcccagcggcggcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgagggggaaggtggcggggtgggaaccgtgtcattccagagtggatctgcggtgaagccaagccgcaaggttacaaggcatcctcaccaggggccccgcctgctgctcccaggtggcctgcggccactgctatgctcaaggacctggaaacccatgcttcgagacaacgtgactttaatgggagggtgggtgggccgcagacaggctggcagggcaggtgctgcgtggggccctctccagcccgtcctaccctgggctcacatggggcctgtgcccacccctcttgagtgtcttggggacagctctttccacccctggaagatggaaataaacctgcgtgtgggtggagtgttctcgtgccgaattcaaaaagctt

SEQ ID NO: 30 - protein, 465 aa, Homo sapiens
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 31 - DNA, 1999 bp, Homo sapiens
  CDS (53)...(1513)
gaattcggcacgagcagaagcaacaataattgtgaaaaatacttcagcagttatggactcatctgtcattcaaaggaaaaaagtagctgtcattggtggtggcttggttggctcattacaagcatgctttcttgcaaagaggaatttccagattgatgtatatgaagctagggaagatactcgagtggctaccttcacacgtggaagaagcattaacttagccctttctcatagaggacgacaagccttgaaagctgttggcctggaagatcagattgtatcccaaggtattcccatgagagcaagaatgatccactctctttcaggaaaaaagtctgcaattccctatgggacaaagtctcagtatattctttctgtaagcagagaaaatctaaacaaggatctattgactgctgctgagaaataccccaatgtgaaaatgcactttaaccacaggctgttgaaatgtaatccagaggaaggaatgatcacagtgcttggatctgacaaagttcccaaagatgtcacttgtgacctcattgtaggatgtgatggagcctattcaactgtcagatctcacctgatgaagaaacctcgctttgattacagtcagcagtacattcctcatgggtacatggagttgactattccacctaagaacggagattatgccatggaacctaattatctgcatatttggcctagaaatacctttatgatgattgcacttcctaacatgaacaaatcattcacatgtactttgttcatgccctttgaagagtttgaaaaacttctaaccagtaatgatgtggtagatttcttccagaaatactttccggatgccatccctctaattggagagaaactcctagtgcaagatttcttcctgttgcctgcccagcccatgatatctgtaaagtgctcttcatttcactttaaatctcactgtgtactgctgggagatgcagctcatgctatagtgccgttttttgggcaaggaatgaatgcgggctttgaagactgcttggtatttgatgagttaatggataaattcagtaacgaccttagtttgtgtcttcctgtgttctcaagattgagaatcccagatgatcacgcgatttcagacctatccatgtacaattacatagagatgcgagcacatgtcaactcaagctggttcatttttcagaagaacatggagagatttcttcatgcgattatgccatcgacctttatccctctctatacaatggtcactttttccagaataagataccatgaggctgtgcagcgttggcattggcaaaaaaaggtgataaacaaaggactctttttcttgggatcactgatagccatcagcagtacctacctacttatacactacatgtcaccacgatctttcctctgcttgagaagaccatggaactggatagctcacttccggaatacaacatgtttccccgcaaaggccgtggactccctagaacaaatttccaatctcattagcaggtgatagaaaggttttgtggtagcaaatgcatgatttctctgtgaccaaaattaagcatgaaaaaaatgtttccattgccatatttgattcactagtggaagatagtgttctgcttataattaaactgaatgtagagtatctctgtatgttaattgcaattactggttggggggtgcattttaaaagatgaaacatgcagcttccctacattacacacactcaggttgagtcattctaactataaaagtgcaatgactaagatccttcacttctctgaaagtaaggccctagatgcctcagggaagacagtaatcatgccttttctttaaaagacacaataggactcgcaacagcattgactcaacacctaggactaaaaatcacaacttaactagcatgttaactgcacttttcattacgtgaatggaacttacctaaccacagggctcagacttactagataaaaccagaaatggaaataaggaattc

SEQ ID NO: 32 - protein, 486 aa, Homo sapiens
MDSSVIQRKKVAVIGGGLVGSLQACFLAKRNFQIDVYEAREDTRVATFTRGRSINLALSHRGRQALKAVGLEDQIVSQGIPMRARMIHSLSGKKSAIPYGTKSQYILSVSRENLNKDLLTAAEKYPNVKMHFNHRLLKCNPEEGMITVLGSDKVPKDVTCDLIVGCDGAYSTVRSHLMKKPRFDYSQQYIPHGYMELTIPPKNGDYAMEPNYLHIWPRNTFMMIALPNMNKSFTCTLFMPFEEFEKLLTSNDVVDFFQKYFPDAIPLIGEKLLVQDFFLLPAQPMISVKCSSFHFKSHCVLLGDAAHAIVPFFGQGMNAGFEDCLVFDELMDKFSNDLSLCLPVFSRLRIPDDHAISDLSMYNYIEMRAHVNSSWFIFQKNMERFLHAIMPSTFIPLYTMVTFSRIRYHEAVQRWHWQKKVINKGLFFLGSLIAISSTYLLIHYMSPRSFLCLRRPWNWIAHFRNTTCFPAKAVDSLEQISNLISR

SEQ ID NO: 33 - DNA, 5415 bp, Homo sapiens
  CDS (173)...(3739)
gggcgcagaggaggaaagggagcaggcgcagggggactggaaaggcagcatgcgctcgccaggagcaacctcggcgcccagggtctgaggctgcagcccagttcgccattgtgagccgccgccgggggagtccgctagcgcacgcgtgcccccgagtccccgtccgcgcacgatggggcacctgcccacggggatacacggcgcccgccgcctcctgcctctgctctggctctttgtgctgttcaagaatgctacagctttccatgtaactgtccaagatgataataacatcgttgtctcattagaagcttcagacgtcatcagtccagcatctgtgtatgttgtgaagataactggtgaatccaaaaattatttcttcgaatttgaggaattcaacagcactttgcctcctcctgttattttcaaggccagttatcatggcctttattatataatcactctggtagtggtaaatggaaatgtggtgaccaagccatccagatcaatcactgtgttaacaaaacctctacctgtaaccagtgtttccatatatgactataaaccttctcctgaaacaggagtcctgtttgaaatacattatccagaaaaatataacgttttcacaagagtgaacattagctactgggaaggtaaagacttccggacaatgctatataaagatttctttaagggaaaaacagtatttaatcactggctgccaggaatgtgttatagtaatatcacctttcagctggtatctgaggcaacttttaataaaagtacccttgttgagtacagtggtgtcagtcacgaacccaaacagcacagaactgccccttatccacctcaaaatatttccgttcgtatcgtaaacttgaacaaaaacaactgggaagaacagagtggcaatttcccagaagaatccttcatgagatcacaagatacaataggaaaagaaaaactcttccattttacagaagaaacccctgaaattccctcgggcaacatttcttccggttggcctgattttaatagcagtgactatgaaactacgtctcagccatattggtgggacagtgcatctgcagctcctgaaagtgaagatgaatttgtcagcgtacttcccatggaatacgaaaataacagtacactcagtgagacagagaagtcaacatcaggctctttctcctttttccctgtgcaaatgatattgacctggttaccacccaaaccacccactgcttttgatgggttccatatccatattgaacgagaagagaactttactgaatatttgatggtggatgaagaagcacatgaatttgttgcagaactgaaggaacctgggaaatataagttatctgtgacaacctttagttcctcaggatcttgtgaaactcgaaaaagtcagtcagcaaaatcactcagcttttatatcagtccttcaggagagtggattgaagaactgaccgagaagccgcagcacgtgagtgtccacgttttaagctcaaccactgccttgatgtcctggacatcttcccaagagaactacaacagcaccattgtgtctgtggtgtcgctgacctgccagaaacaaaaggagagccagaggcttgaaaagcagtactgcactcaggtgaactcaagcaaacctattattgaaaatctggttcctggtgcccagtaccaggttgtaatatacctaaggaaaggccctttgattggaccaccttcagatcctgtgacatttgctattgttcccacaggaataaaggatttaatgctctatcctttgggtcctacggccgtggttctgagctggaccagaccttatttaggcgtgttcagaaaatacgtggttgaaatgttttatttcaaccctgctacaatgacatcagagtggaccacctactatgaaatagcagcaactgtttccttaactgcatccgtgagaatagctaatctgctgccagcatggtactacaacttccgggttaccatggtgacgtggggagatccagaattgagctgctgtgacagctctaccatcagcttcataacagccccagtggctccggaaatcacttctgtggaatatttcaacagtctgttatatatcagttggacatatggggatgatacaacggacttgtcccattctagaatgcttcactggatggtggttgcagaaggaaaaaagaaaattaaaaagagtgtaacacgcaatgtcatgactgcaattctcagcttgcctccaggcgacatctataacctctcagtaactgcttgtactgaaagaggaagtaatacctccatgctccgccttgtcaagctagaaccagctccacccaaatcactcttcgcagtgaacaaaacccagacttcagtgactttgctgtgggtggaagagggagtagctgatttctttgaagttttctgtcaacaagttggctccagtcagaaaaccaaacttcaggaaccagttgctgtttcttcccatgtcgtgaccatctccagccttcttcctgccactgcctacaattgtagtgtcaccagctttagccatgacagccccagtgtccctacgttcatagccgtctcaacaatggttacagagatgaatcccaatgtggtagtgatctccgtgctggccatccttagcacacttttaattggactgttgcttgttaccctcattattcttaggaaaaagcatctgcagatggctagggagtgtggagctggtacatttgtcaattttgcatccttagagagggatggaaagcttccatacaactggagtaaaaatggtttaaagaagaggaaactgacaaacccggttcaactggatgactttgatgcctatattaaggatatggccaaagactctgactataaattttctcttcagtttgaggagttgaaattgattggactggatatcccacactttgctgcagatcttccactgaatcgatgtaaaaaccgttacacaaacatcctaccatatgacttcagccgtgtgagattagtctccatgaatgaagaggaaggtgcagactacatcaatgccaactatattcctggatacaactcaccccaggagtatattgccacccaggggccactgcctgaaaccagaaatgacttctggaagatggtcctgcaacaaaagtctcagattattgtcatgctcactcagtgtaatgagaaaaggagggtgaaatgtgaccattactggccattcacggaagaacctatagcctatggagacatcactgtggagatgatttcagaggaagagcaggacgactgggcctgtagacacttccggatcaactatgctgacgagatgcaggatgtgatgcattttaactacactgcatggcctgatcatggtgtgcccacagcaaatgctgcagaaagtatcctgcagtttgtacacatggtccgacagcaagctaccaagagcaaaggtcccatgatcattcactgcagtgctggcgtgggacggacaggaacattcattgccctggacaggctcttgcagcacattcgggatcatgagtttgttgacatcttagggctggtgtcagaaatgaggtcataccggatgtctatggtacagacagaggagcagtacatttttatccatcagtgtgtgcaactgatgtggatgaagaagaagcagcagttctgcatcagtgatgtcatatacgagaatgttagcaagtcctagttcagaatccggagcagagaggacatgatgtgcgcccatcctcccttgcttccagattgttttagtgggccctgatggtcatttttctaaacagaggccctgctttgtaatatgtggccaaggagataatttatctcacagaagcaccgggaagacttagccttaaagagcctacagtgtccttttggactctttcacttcgggacatttaataatggaccaaattcaacagaacaccaggaaggtcaagacgctctccaaagggcaggaagtacagcacttccgaagagtttagttggccctttgctggttgggctgagttttttatttttaagtgtttgtttttcagtgcaataatttttgtgtgtgtgtgattcttatcagaaagttgaattgttttctgcctacaccgttcatcagccccataacccaggaaggaacaggcattgttagcatcagattatacctcattattaaaaggaggcatggccacacatgaagaaatggtcattctacttcaaagaaattgagccagcactatctgtactccaacattaccggatctggattggggaggttggtcagggaagagaggggttctacccacagatcaactgtgtaatcttttactattcaagctataattcagcttcaaagtagagtagaaaaaaaattgtcttaactgttctagttcttgatggttttcttccttattaacagttggtgtttcttccttggcccttttggactaatgttactgtccaagttctttctcaagaaaccacatctggttcagaagagtgtcaagttggactctttgaactctgttgctgtctgagcaatcgtggtgcctagactttgcattccttgttctgttgacctgcatacatgtgagagctatttctttaagaactatataggctgtgaaaacgcactttctttcccccaaagagctgggaatttatgaagttatggcaatgaactgcagcatgctgggacaattatttgactacttttttttgtaatattgtcaaatgtctctatggattctgacagagatttctttttgttttgttattcttttggttgtcagtttcattttaacgagtgtaactagtaacattttattctttggattttgtataattacagtacatgattgtgtattgtgacatgaatgctgtcaaaatgacattgatggcattgtgaagcctgttactttgtgtcacttcctgataaataagaggtgatgacatggatatacaacagaaaacactttgagttgaaagtaaacacaagctggctgcttccctgtggcaaaaaaaaaaaaaatagcattcctgcacatctgtacccacgccttcttcaaagccatactatttatgtgctccggatccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttcccc

SEQ ID NO: 34 - protein, 1188 aa, Homo sapiens
MGHLPTGIHGARRLLPLLWLFVLFKNATAFHVTVQDDNNIVVSLEASDVISPASVYVVKITGESKNYFFEFEEFNSTLPPPVIFKASYHGLYYIITLVVVNGNVVTKPSRSITVLTKPLPVTSVSIYDYKPSPETGVLFEIHYPEKYNVFTRVNISYWEGKDFRTMLYKDFFKGKTVFNHWLPGMCYSNITFQLVSEATFNKSTLVEYSGVSHEPKQHRTAPYPPQNISVRIVNLNKNNWEEQSGNFPEESFMRSQDTIGKEKLFHFTEETPEIPSGNISSGWPDFNSSDYETTSQPYWWDSASAAPESEDEFVSVLPMEYENNSTLSETEKSTSGSFSFFPVQMILTWLPPKPPTAFDGFHIHIEREENFTEYLMVDEEAHEFVAELKEPGKYKLSVTTFSSSGSCETRKSQSAKSLSFYISPSGEWIEELTEKPQHVSVHVLSSTTALMSWTSSQENYNSTIVSVVSLTCQKQKESQRLEKQYCTQVNSSKPIIENLVPGAQYQVVIYLRKGPLIGPPSDPVTFAIVPTGIKDLMLYPLGPTAVVLSWTRPYLGVFRKYVVEMFYFNPATMTSEWTTYYEIAATVSLTASVRIANLLPAWYYNFRVTMVTWGDPELSCCDSSTISFITAPVAPEITSVEYFNSLLYISWTYGDDTTDLSHSRMLHWMVVAEGKKKIKKSVTRNVMTAILSLPPGDIYNLSVTACTERGSNTSMLRLVKLEPAPPKSLFAVNKTQTSVTLLWVEEGVADFFEVFCQQVGSSQKTKLQEPVAVSSHVVTISSLLPATAYNCSVTSFSHDSPSVPTFIAVSTMVTEMNPNVVVISVLAILSTLLIGLLLVTLIILRKKHLQMARECGAGTFVNFASLERDGKLPYNWSKNGLKKRKLTNPVQLDDFDAYIKDMAKDSDYKFSLQFEELKLIGLDIPHFAADLPLNRCKNRYTNILPYDFSRVRLVSMNEEEGADYINANYIPGYNSPQEYIATQGPLPETRNDFWKMVLQQKSQIIVMLTQCNEKRRVKCDHYWPFTEEPIAYGDITVEMISEEEQDDWACRHFRINYADEMQDVMHFNYTAWPDHGVPTANAAESILQFVHMVRQQATKSKGPMIIHCSAGVGRTGTFIALDRLLQHIRDHEFVDILGLVSEMRSYRMSMVQTEEQYIFIHQCVQLMWMKKKQQFCISDVIYENVSKS

SEQ ID NO: 35 - DNA, 2373 bp, Homo sapiens
  CDS (6)...(2129)
tcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 36 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 37 - DNA, 934 bp, Homo sapiens
  CDS (8)...(751)
tgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctctcccagtgtaaatctggagccaagtccagatttacactgggagaggtgccagcaactgaataaatacctcttagctgagtgg

SEQ ID NO: 38 - protein, 247 aa, Homo sapiens
MQPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 39 - DNA, 1853 bp, Homo sapiens
  CDS (27)...(1814)
aaaacgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgcatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccatcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcaccgtggctcacgtctgtaatcccacctttt

SEQ ID NO: 40 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCHPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 41 - DNA, 1700 bp, Homo sapiens
  CDS (125)...(1585)
cggaattggtgggttcttggtctcactgagttctagaatgaagctgcagaccctcgcagtgagtgttacagctcttaaggctctctgactgccacccctgcctgcctgcccggccctgcacaacatgcagccctccggcctcgagggtcccggcacgtttggtcggtggcctctgctgagtctgctgctcctgctgctgctgctccagcctgtaacctgtgcctacaccacgccaggcccccccagagccctcaccacgctgggcgcccccagagcccacaccatgccgggcacctacgctccctcgaccacactcagtagtcccagcacccagggcctgcaagagcaggcacgggccctgatgcgggacttcccgctcgtggacggccacaacgacctgcccctggtcctaaggcaggtttaccagaaagggctacaggatgttaacctgcgcaatttcagctacggccagaccagcctggacaggcttagagatggcctcgtgggcgcccagttctggtcagcctatgtgccatgccagacccaggaccgggatgccctgcgcctcaccctggagcagattgacctcatacgccgcatgtgtgcctcctattctgagctggagcttgtgacctcggctaaagctctgaacgacactcagaaattggcctgcctcatcggtgtagagggtggccactcgctggacaatagcctctccatcttacgtaccttctacatgctgggagtgcgctacctgacgctcacccacacctgcaacacaccctgggcagagagctccgctaagggcgtccactccttctacaacaacatcagcgggctgactgactttggtgagaaggtggtggcagaaatgaaccgcctgggcatgatggtagacttatcccatgtctcagatgctgtggcacggcgggccctggaagtgtcacaggcacctgtgatcttctcccactcggctgcccggggtgtgtgcaacagtgctcggaatgttcctgatgacatcctgcagcttctgaagaagaacggtggcgtcgtgatggtgtctttgtccatgggagtaatacagtgcaacccatcagccaatgtgtccactgtggcagatcacttcgaccacatcaaggctgtcattggatccaagttcatcgggattggtggagattatgatggggccggcaaattccctcaggggctggaagacgtgtccacatacccggtcctgatagaggagttgctgagtcgtggctggagtgaggaagagcttcagggtgtccttcgtggaaacctgctgcgggtcttcagacaagtggaaaaggtacaggaagaaaacaaatggcaaagccccttggaggacaagttcccggatgagcagctgagcagttcctgccactccgacctctcacgtctgcgtcagagacagagtctgacttcaggccaggaactcactgagattcccatacactggacagccaagttaccagccaagtggtcagtctcagagtcctccccccacatggccccagtccttgcagttgtggccaccttcccagtccttattctgtggctctgatgacccagttagtcctgccagatgtcactgtagcaagccacagacaccccacaaagttcccctgttgtgcaggcacaaatatttcttgaaataaatgttttggacatagaaaaaa

SEQ ID NO: 42 - protein, 486 aa, Homo sapiens
MQPSGLEGPGTFGRWPLLSLLLLLLLLQPVTCAYTTPGPPRALTTLGAPRAHTMPGTYAPSTTLSSPSTQGLQEQARALMRDFPLVDGHNDLPLVLRQVYQKGLQDVNLRNFSYGQTSLDRLRDGLVGAQFWSAYVPCQTQDRDALRLTLEQIDLIRRMCASYSELELVTSAKALNDTQKLACLIGVEGGHSLDNSLSILRTFYMLGVRYLTLTHTCNTPWAESSAKGVHSFYNNISGLTDFGEKVVAEMNRLGMMVDLSHVSDAVARRALEVSQAPVIFSHSAARGVCNSARNVPDDILQLLKKNGGVVMVSLSMGVIQCNPSANVSTVADHFDHIKAVIGSKFIGIGGDYDGAGKFPQGLEDVSTYPVLIEELLSRGWSEEELQGVLRGNLLRVFRQVEKVQEENKWQSPLEDKFPDEQLSSSCHSDLSRLRQRQSLTSGQELTEIPIHWTAKLPAKWSVSESSPHMAPVLAVVATFPVLILWL

SEQ ID NO: 43 - DNA, 2850 bp, Homo sapiens
  CDS (81)...(1826)
ccggctcggtaccactataacggccgccagtgtgctggaattcgcccttgcgcagatcgctggctgcagttggcgggcgcatgtgggggcgcacggcgcggcggcgctgcccgcgggaactgcggcgcggccgggaggcgctgttggtgctcctggcgctactggcgttggccgggctgggctcggtgctgcgggcgcagcgtggggccggggccggggctgccgagccgggacccccgcgcaccccgcgccccgggcggcgcgagccggtcatgccgcggccgccggtgccggcgaacgcgctgggcgcgcggggcgaggcggtgcggctgcagctgcagggcgaggagctgcggctgcaggaggagagcgtgcggctgcaccagattaacatctacctcagcgaccgcatctcactgcaccgccgcctgcccgagcgctggaacccgctgtgcaaagagaagaaatatgattatgataatttgcccaggacatctgttatcatagcattttataatgaagcctggtcaactctccttcggacagtttacagtgtccttgagacatccccggatatcctgctagaagaagtgatccttgtagatgactacagtgatagagagcacctgaaggagcgcttggccaatgagctttcgggactgcccaaggtgcgcctgatccgcgccaacaagagagagggcctggtgcgagcccggctgctgggggcgtctgcggcgaggggcgatgttctgaccttcctggactgtcactgtgagtgccacgaagggtggctggagccgctgctgcagaggatccatgaagaggagtcggcagtggtgtgcccggtgattgatgtgatcgactggaacaccttcgaatacctggggaactccggggagccccagatcggcggtttcgactggaggctggtgttcacgtggcacacagttcctgagagggagaggatacggatgcaatcccccgtcgatgtcatcaggtctccaacaatggctggtgggctgtttgctgtgagtaagaaatattttgaatatctggggtcttatgatacaggaatggaagtttggggaggagaaaacctcgaattttcctttaggatctggcagtgtggtggggttctggaaacacacccatgttcccatgttggccatgttttccccaagcaagctccctactcccgcaacaaggctctggccaacagtgttcgtgcagctgaagtatggatggatgaatttaaagagctctactaccatcgcaacccccgtgcccgcttggaaccttttggggatgtgacagagaggaagcagctccgggacaagctccagtgtaaagacttcaagtggttcttggagactgtgtatccagaactgcatgtgcctgaggacaggcctggcttcttcgggatgctccagaacaaaggactaacagactactgctttgactataaccctcccgatgaaaaccagattgtgggacaccaggtcattctgtacctctgtcatgggatgggccagaatcagtttttcgagtacacgtcccagaaagaaatacgctataacacccaccagcctgagggctgcattgctgtggaagcaggaatggatacccttatcatgcatctctgcgaagaaactgccccagagaatcagaagttcatcttgcaggaggatggatctttatttcacgaacagtccaagaaatgtgtccaggctgcgaggaaggagtcgagtgacagtttcgttccactcttacgagactgcaccaactcggatcatcagaaatggttcttcaaagagcgcatgttatgaagcctcgtgtatcaaggagcccatcgaaggagactgtggagccaggactctgcccaacaaagacttagctaagcagtgaccagaacccaccaaaaactaggctgcattgctttgaagaggcaatcattttgccatttgtgaaagttgtgttggatttagtaaaaatgtgaataagctttgtacttattttgagaactttttaaatgttccaaaataccctattttcaaagggtaatcgtaagatgttaacccttggtatttagaaaattaaaaccttataatatttttctatcaarawrwawattttacagtcgtgccttttactctcattagcaaaaaagataaagattttattttggtatttacaagaattcccaggtacgaagatatctgcatgggtggaaatcaggttcaagcaacgtactttgcattaactgataatacctcagctgcggggttaaagttttcccagtatagagagactgtcactaggaacattgtattgatttattcaggtcattgagatcttctagatgtattttaaaaagaatgctttttggttatgtgttgctaccacagttaacactccataatgttcatgtcagccaaagaggactaaccaaagctgaaatctcagagaacaatttgctttactaagctgagtcaacttgagagcgaacttctaacaatgccgcactgtagtgtggctggttctaccactatgactttaaaacatgtttatatcatttttaatttttatgatacggtagtgtcagggagaaatgtaatgttctatatgaaattcctttttcaagtttgttcattaataacagttattaatttaaatcagcgttagagtttgtgctgctgcaactgctgtgaaaatttctctgagtaattctgatttgtgaatgatcccagaccaaccctgagattttgtcaacctgattaagtcaatatgaatgattaaaaagatgtgagaacaaaaaaaaaaaaaaaaaaaaaaaaaaaaaaaaaaaaaaaa

SEQ ID NO: 44 - protein, 581 aa, Homo sapiens [sequence omitted]

SEQ ID NO: 45 - DNA, 2890 bp, Homo sapiens
  CDS (229)...(1116)
gtcgcagattcaaacaaatagcagcgaacagggaatgacagttccaccagaagacgattaagccacagcctctaattggaacggcatttgtacagtcagagactcttaccagacatctccaggaatctgtgagccattgtcaaaacgtccattttcatctggctgtgaaagtgaggaccacaacaggtaggtattggtagaaacaggagtcctcagagaagccccaagatgcagcctgagggagcagaaaagggaaaaagcttcaagcagagactggtcttgaagagcagcttagcgaaagaaaccctctctgagttcttgggcacgttcatcttgattgtccttggatgtggctgtgttgcccaagctattctcagtcgaggacgttttggaggggtcatcactatcaatgttggattttcaatggcagttgcaatggccatttatgtggctggcggtgtctctggtggtcacatcaacccagctgtgtctttagcaatgtgtctctttggacggatgaaatggttcaaattgccattttatgtgggagcccagttcttgggagcctttgtgggggctgcaaccgtctttggcatttactatgatggacttatgtcctttgctggtggaaaactgctgatcgtgggagaaaatgcaacagcacacatttttgcaacatacccagctccgtatctatctctggcgaacgcatttgcagatcaagtggtggccaccatgatactcctcataatcgtctttgccatttttgactccagaaacttgggagcccccagaggcctagagcccattgccatcggcctcctgattattgtcattgcttcctccctgggactgaacagtggctgtgccatgaacccagctcgagacctgagtcccagacttttcactgccttggcaggctgggggtttgaagtcttcagagctggaaacaacttctggtggattcctgtagtgggccctttggttggtgctgtcattggaggcctcatctatgttcttgtcattgaaatccaccatccagagcctgactcagtctttaaggcagaacaatctgaggacaaaccagagaaatatgaactcagtgtcatcatgtagtggcatgctcagctctggatttgcagtcagtttgggattctcttcagaaagatggcatctaagtgtctgtgttcttgtaagcctgaggtggaatccacccagttttgtctgctagccatatgggacatctaattggaaaagcatctgcataaaagtttggaaacaatgaccacttctctaccattgtcccccaccccaccccccagaataacgctgactgtcccctgaaacagccttctctcctgccctgtttatttcatcctcgatgggaattcttgctaggtaagcactaataactcggcatcttgacgatagtcccatttgggtggtttcagctgcactatctgtatgaaatggtgtcaccaaaacccttttcttcagtatcgacaaagattacattctgagtaccaaccaaaccctaaattgaaagacaaaactatggtttcagtcaacatattcatgaattagggagctaatgggttaagcttccagttcccgctatgctactggatttgtataaatactgatattctccaaacctagtggtgtagggagcaagagaatgcagctggaaggcacaaggggaggacattgtggcattcagaaactgcaggagacaagatgaatttgagaagccaaatggaatttttaatggaaaccatttatcagattaatctcttgctctcctgcattttagaggacaccaattaatttcctggtctttagtatataataacctaaaataccattgtaacctcagtcatgaaaaatacatcactctgtctttttagctcaaatgtattttcctaattgcccacttgagaacagacatttgacaagttatatcaacgactgtgcttgtccattattttacacatgccctagaagccaaaactgaaagccactggatcctggtctagctgaatcttcagagtgggaggtctccaaaaagatattaccttattgggcttaacaattcacaaggcactttcacacccattatctaatttaatcctcataatgactatgtgaggcaaatgccacattgcccatttttcagataaagaaacaaaatcttagggaagataagttgagttgtccaagagcacactgaaagttgaatgttatctaatgcattcctctacctttcagaagatcagtagctggctgagaatctttgccaaatcttccttgctagccagaagtggaattggcagcttctagaatatgtacacctctggacaaaatgttcctcaatcttaagatacaaagaccctcattgtctgggtctattcccacacttactgagtacagatgaaggaaagtggtagcaatttaatcataactttcatttgctgaaaaacattatgagaaggcctcccttcctaagccacctctggtcttgctaagtcttgatcttgcttcctgccagcaccaaacattacattcaggggatttcctctggctcagtcttttccccttgaagttctctaatagatgttacttttgacaaaagatcgcctatgagttacaagcaccaggggatgctctacatcaagggatgcaccttcagtcaaactgtcaaaaagcccagaattcccaaaggcattaggtttcccaactgctttgtgctgatatcagaacagcagaaattaaatgtgaaatgtttctgatgacttatgttctacaatctatggacatacgggatttttttttcttgctttgaagctacctggatatttcctatttgaaataaaattgttcggtcattgtt

SEQ ID NO: 46 - protein, 295 aa, Homo sapiens
MQPEGAEKGKSFKQRLVLKSSLAKETLSEFLGTFILIVLGCGCVAQAILSRGRFGGVITINVGFSMAVAMAIYVAGGVSGGHINPAVSLAMCLFGRMKWFKLPFYVGAQFLGAFVGAATVFGIYYDGLMSFAGGKLLIVGENATAHIFATYPAPYLSLANAFADQVVATMILLIIVFAIFDSRNLGAPRGLEPIAIGLLIIVIASSLGLNSGCAMNPARDLSPRLFTALAGWGFEVFRAGNNFWWIPVVGPLVGAVIGGLIYVLVIEIHHPEPDSVFKAEQSEDKPEKYELSVIM

SEQ ID NO: 47 - DNA, 1869 bp, Homo sapiens
  CDS (1)...(1869)
atgaggctcctccgcagacgccacatgcccctgcgcctggccatggtgggctgcgcctttgtgctcttcctcttcctcctgcatagggatgtgagcagcagagaggaggccacagagaagccgtggctgaagtccctggtgagccggaaggatcacgtcctggacctcatgctggaggccatgaacaaccttagagattcaatgcccaagctccaaatcagggctccagaagcccagcagactctgttctccataaaccagtcctgcctccctgggttctataccccagctgaactgaagcccttctgggaacggccaccacaggaccccaatgcccctggggcagatggaaaagcatttcagaagagcaagtggacccccctggagacccaggaaaaggaagaaggctataagaagcactgtttcaatgcctttgccagcgaccggatctccctgcagaggtccctggggccagacacccgaccacctgagtgtgtggaccagaagttccggcgctgccccccactggccaccaccagcgtgatcattgtgttccacaacgaagcctggtccacactgctgcgaacagtgtacagcgtcctacacaccacccctgccatcttgctcaaggagatcatactggtggatgatgccagcacagaggagcacctaaaggagaagctggagcagtacgtgaagcagctgcaggtggtgagggtggtgcggcaggaggagcggaaggggttgatcaccgcccggctgctgggggccagcgtggcacaggcggaggtgctcacgttcctggatgcccactgtgagtgcttccacggctggctggagcccctcctggctcgaatcgctgaggacaagacagtggtggtgagcccagacatcgtcaccatcgaccttaatacttttgagttcgccaagcccgtccagaggggcagagtccatagccgaggcaactttgactggagcctgaccttcggctgggaaacacttcctccacatgagaagcagaggcgcaaggatgaaacataccccatcaaatccccgacgtttgctggtggcctcttctccatccccaagtcctactttgagcacatcggtacctatgataatcagatggagatctggggaggggagaacgtggaaatgtccttccgggtgtggcagtgtgggggccagctggagatcatcccctgctctgtcgtaggccatgtgttccggaccaagagcccccacaccttccccaagggcactagtgtcattgctcgcaatcaagtgcgcctggcagaggtctggatggacagctacaagaagattttctataggagaaatctgcaggcagcaaagatggcccaagagaaatccttcggtgacatttcggaacgactgcagctgagggaacaactgcactgtcacaacttttcctggtacctgcacaatgtctacccagagatgtttgttcctgacctgacgcccaccttctatggtgccatcaagaacctcggcaccaaccaatgcctggatgtgggtgagaacaaccgcggggggaagcccctcatcatgtactcctgccacggccttggcggcaaccagtactttgagtacacaactcagagggaccttcgccacaacatcgcaaagcagctgtgtctacatgtcagcaagggtgctctgggccttgggagctgtcacttcactggcaagaatagccaggtccccaaggacgaggaatgggaattggcccaggatcagctcatcaggaactcaggatctggtacctgcctgacatcccaggacaaaaagccagccatggccccctgcaatcccagtgacccccatcagttgtggctctttgtctag

SEQ ID NO: 48 - protein, 622 aa, Homo sapiens
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSIPKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 49 - DNA, 1544 bp, Homo sapiens
  CDS (87)...(1517)
gtggccgggagaaagtgtctctcatttaggagggtttgcaggtccagagtaaagtcactgaagagtggaagcgaggaaggaacaggatgattagacctcagctgcggaccgcggggctgggacgatgcctcctgccggggctgctgctgctcctggtgcccgtcctctgggccggggctgaaaagctacatacccagccctcctgccccgcggtctgccagcccacgcgctgccccgcgctgcccacctgcgcgctggggaccacgccggtgttcgacctgtgccgctgttgccgcgtctgccccgcggccgagcgtgaagtctgcggcggggcgcagggccaaccgtgcgccccggggctgcagtgcctccagccgctgcgccccgggttccccagcacctgcggttgcccgacgctgggaggggccgtgtgcggcagcgacaggcgcacctaccccagcatgtgcgcgctccgggccgaaaaccgcgccgcgcgccgcctgggcaaggtcccggccgtgcctgtgcagtgggggaactgcggggatacagggaccagaagcgcaggcccgctcaggaggaattacaacttcatcgccgcggtggtggagaaggtggcgccatcggtggttcacgtgcagctgtggggcaggttacttcacggcagcaggcttgttcctgtgtacagtggctctgggttcatagtgtctgaggacgggctcattattaccaatgcccatgttgtcaggaaccagcagtggattgaggtggtgctccagaatggggcccgttatgaagctgttgtcaaggatattgaccttaaattggatcttgcggtgattaagattgaatcaaatgctgaacttcctgtactgatgctgggaagatcatctgaccttcgggctggagagtttgtggtggctttgggcagcccattttctctgcagaacacagctactgcaggaattgtcagcaccaaacagcgagggggcaaagaactggggatgaaggattcagatatggactacgtccagattgatgccacaattaactatgggaattctggtggtcctctggtgaacttggatggtgatgtgattggcgtcaattcattgagggtgactgatggaatctcctttgcaattccttcagatcgagttaggcagttcttggcagaataccatgagcaccagatgaaaggaaaggcgttttcaaataagaaatatctgggtctgcaaatgctgtccctcactgtgccccttagtgaagaattgaaaatgcattatccagatttccctgatgtgagttctggggtttatgtatgtaaagtggttgaaggaacagctgctcaaagctctggattgagagatcacgatgtaattgtcaacataaatgggaaacctattactactacaactgatgttgttaaagctcttgacagtgattccctttccatggctgttcttcggggaaaagataatttgctcctgacagtcatacctgaaacaatcaattaaatatcttgttttaaagtgggcttatct

SEQ ID NO: 50 - protein, 476 aa, Homo sapiens
MIRPQLRTAGLGRCLLPGLLLLLVPVLWAGAEKLHTQPSCPAVCQPTRCPALPTCALGTTPVFDLCRCCRVCPAAEREVCGGAQGQPCAPGLQCLQPLRPGFPSTCGCPTLGGAVCGSDRRTYPSMCALRAENRAARRLGKVPAVPVQWGNCGDTGTRSAGPLRRNYNFIAAVVEKVAPSVVHVQLWGRLLHGSRLVPVYSGSGFIVSEDGLIITNAHVVRNQQWIEVVLQNGARYEAVVKDIDLKLDLAVIKIESNAELPVLMLGRSSDLRAGEFVVALGSPFSLQNTATAGIVSTKQRGGKELGMKDSDMDYVQIDATINYGNSGGPLVNLDGDVIGVNSLRVTDGISFAIPSDRVRQFLAEYHEHQMKGKAFSNKKYLGLQMLSLTVPLSEELKMHYPDFPDVSSGVYVCKVVEGTAAQSSGLRDHDVIVNINGKPITTTTDVVKALDSDSLSMAVLRGKDNLLLTVIPETIN

SEQ ID NO: 51 - DNA, 2187 bp, Homo sapiens
  CDS (61)...(1473)
cgcccgggcaggtgagaaattggagaagataaaactggacactggggagaccacaacttcatgctgcgtgggatctcccagctacctgcagtggccaccatgtcttgggtcctgctgcctgtactttggctcattgttcaaactcaagcaatagccataaagcaaacacctgaattaacgctccatgaaatagtttgtcctaaaaaacttcacattttacacaaaagagagatcaagaacaaccagacagaaaagcatggcaaagaggaaaggtatgaacctgaagttcaatatcagatgatcttaaatggagaagaaatcattctctccctacaaaaaaccaagcacctcctggggccagactacactgaaacattgtactcacccagaggagaggaaattaccacgaaacctgagaacatggaacactgttactataaaggaaacatcctaaatgaaaagaattctgttgccagcatcagtacttgtgacgggttgagaggatacttcacacatcatcaccaaagataccagataaaacctctgaaaagcacagacgagaaagaacatgccgtctttacatctaaccaggaggaacaagacccagctaaccacacatgtggtgtgaagagcactgacgggaaacaaggcccaattcgaatctctagatcactcaaaagcccagagaaagaagactttcttcgggcacagaaatacattgatctctatttggtgctggataatgccttttataagaactataatgagaatctaactctgataagaagctttgtgtttgatgtgatgaacctactcaatgtgatatataacaccatagatgttcaagtggccttggtaggtatggaaatctggtctgatggggataagataaaggtggtgcccagcgcaagcaccacgtttgacaacttcctgagatggcacagttctaacctggggaaaaagatccacgaccatgctcagcttctcagcgggattagcttcaacaatcgacgtgtgggactggcagcttcaaattccttgtgttccccatcttcggttgctgttattgaggctaaaaaaaagaataatgtggctcttgtaggagtgatgtcacatgagctgggccatgtccttggtatgcctgatgttccattcaacaccaagtgtccctctggcagttgtgtgatgaatcagtatctgagttcaaaattcccaaaggatttcagtacatcttgccgtgcacattttgaaagataccttttatctcagaaaccaaagtgcctgctgcaagcacctattcctacaaatataatgacaacaccagtgtgtgggaaccaccttctagaagtgggagaagactgtgattgtggctctcctaaggagtgtaccaatctctgctgtgaagccctaacgtgtaaactgaagcctggaactgattgcggaggagatgctccaaaccataccacagagtgaatccaaagtctgcttcactgagatgctaccttgccaggacaagaaccaagaactctaactgtcccaggaatcttgtgaattttcacccataatggtctttcacttgtcattctactttctatattgttatcagtccaggaaacaggtaaacagatgtaattagagacattggctctttgtttaggcctaatctttctttttacttttttttttcttttttctttttttttaaagatcatgaatttgtgacttagttctgccctttggagaacaaaagaaagcagtcttccatcaaatcaccttaaaatgcacggctaaactattcagagttaacactccagaattgttaaattacaagtactatgctttaatgcttctttcatcttactagtatggcctataaaaaaaataataccacttgatgggtgaaggctttggcaatagaaagaagaatagaattcaggttttatgttattcctctgtgttcacttcgccttgctcttgaaagtgcagtatttttctacatcatgtcgagaatgattcaatgtaaatatttttcattttatcatgtatatcctatacacacatctccttcatcatcatatatgaagtttattttgagaagtctacattgcttacattttaattgagccagcaaagaaggcttaatgatttattgaaccataatgtcaataaaaacacaacttttgaggc

SEQ ID NO: 52 - protein, 470 aa, Homo sapiens
MLRGISQLPAVATMSWVLLPVLWLIVQTQAIAIKQTPELTLHEIVCPKKLHILHKREIKNNQTEKHGKEERYEPEVQYQMILNGEEIILSLQKTKHLLGPDYTETLYSPRGEEITTKPENMEHCYYKGNILNEKNSVASISTCDGLRGYFTHHHQRYQIKPLKSTDEKEHAVFTSNQEEQDPANHTCGVKSTDGKQGPIRISRSLKSPEKEDFLRAQKYIDLYLVLDNAFYKNYNENLTLIRSFVFDVMNLLNVIYNTIDVQVALVGMEIWSDGDKIKVVPSASTTFDNFLRWHSSNLGKKIHDHAQLLSGISFNNRRVGLAASNSLCSPSSVAVIEAKKKNNVALVGVMSHELGHVLGMPDVPFNTKCPSGSCVMNQYLSSKFPKDFSTSCRAHFERYLLSQKPKCLLQAPIPTNIMTTPVCGNHLLEVGEDCDCGSPKECTNLCCEALTCKLKPGTDCGGDAPNHTTE

SEQ ID NO: 53 - protein, 6 aa, Artificial Sequence
  Synthetic peptide
WKYMVM

(19) United States
(12) Patent Application Publication
(10) Pub. No.: US 2004/0180332 A1
(43) Pub. Date: Sep. 16, 2004

(54) Methods and compositions for treating aids and HIV-related disorders using 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777

(75) Inventor: Douglas M. Powell, Littleton, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/789,241

(22) Filed: Feb. 27, 2004

Related U.S. Application Data
(60) Provisional application No. 60/454,202, filed on Mar. 12, 2003.
(60) Provisional application No. 60/456,326, filed on Mar. 20, 2003.
(60) Provisional application No. 60/465,240, filed on Apr. 24, 2003.
(60) Provisional application No. 60/475,233, filed on Jun. 2, 2003.
(60) Provisional application No. 60/478,952, filed on Jun. 16, 2003.
(60) Provisional application No. 60/487,836, filed on Jul. 16, 2003.
(60) Provisional application No. 60/500,111, filed on Sep. 4, 2003.

(51) Int. Cl. C12Q 1/70

(52) U.S. Cl. 435/5

(57) ABSTRACT

The present invention relates to methods for the diagnosis and treatment of AIDS or an HIV-related disorder or disorders. Specifically, the present invention identifies the differential expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 and 46777 genes in tissues relating to AIDS or an HIV-related disorder, relative to their expression in normal, or non-AIDS or HIV-related disease states, and/or in response to manipulations relevant to AIDS or an HIV-related disorder. The present invention describes methods for the diagnostic evaluation and prognosis of various HIV-related disorders, and for the identification of subjects exhibiting a predisposition to such conditions. The invention also provides methods for identifying a compound capable of modulating AIDS or an HIV-related disorder or disorders. The present invention also provides methods for the identification and therapeutic use of compounds as treatments of AIDS or an HIV-related disorder.

### RELATED APPLICATIONS

The present application claims the benefit of U.S. Provisional Application Serial No. 60/454, 202, filed on Mar. 12, 2003, of U.S. Provisional Application Serial No. 60/456, 326, filed on Mar. 20, 2003, of U.S. Provisional Application Serial No. 60/465, 240, filed on Apr. 24, 2003, of U.S. Provisional Application Serial No. 60/475, 233, filed on Jun. 2, 2003, of U.S. Provisional Application Serial No. 60/478, 952, filed on Jun. 16, 2003, of U.S. Provisional Application Serial No. 60/487, 836, filed on Jul. 16, 2003, and of U.S. Provisional Application Serial No. 60/500, 111, filed on Sep. 4, 2003. The entire contents of these provisional patent applications are hereby incorporated in their entirety by this reference.

### BACKGROUND OF THE INVENTION

Human Immunodeficiency Virus (HIV) is a member the lentivirus genus of the Retroviridae family. On the basis of serologic properties and sequence analysis of molecularly cloned genomes human lentivirus isolates are designated HIV-1 and HIV-2. A classification scheme based on the sequence of the viral envelope (env) protein recognizes several subtypes/clades (e.g. HIV-1 A-I). Viral diversification is a key feature of HIV phylogeny. Each subtype displays a high degree of variability. Mutations introduced by the error-prone viral reverse transcriptase represent the major factor for variation, but also recombination occurs within individuals infected with different clades. Molecular epidemiology studies indicate, that viral migration/trafficking rather than viral mutation is the ecologic driving force for the pattern of global variation and distribution.

HIV represents an enveloped virus with two identical copies of a (+)-stranded RNA genome of 9.2 kb in length coding for 9 structural and regulatory viral proteins. Initial steps of infection are mediated through specific interaction of the viral envelope glycoprotein and the major host cell receptor CD4 as well as specific coreceptors CXCR4 (T-troph)/CCR5 (M-troph). After penetration virion RNA is converted into double-stranded DNA by the viral reverse transcriptase. Concomitantly, viral integrase and host cell proteins carry out integration of the linear DNA into the host cell genome to produce the provirus. This intracellular genomic form represents the template for synthesis of full length genomic or subgenomic (spliced and unspliced forms) single-stranded viral RNAs catalyzed by the cellular RNA polymerase II.

HIV encodes precursor polyproteins as well as additional open reading frames. The gag, pol and env genes encode precursors for the virion capsid proteins, several virion enzymes (protease, reverse transcriptase/RNAse H, integrase) as well as the envelope glycoprotein, respectively. The transcriptional activator (tat) and regulator of viral transcription (rev) encode nonstructural essential proteins. In contrast vif, vpr (HIV-1), vpu (HIV-2) and nef encoded genes represent nonessential ‘accessory’ proteins, which are thought to exert their pleiotrophic regulatory/modulatory effects through specific interactions with several different host cell encoded proteins.

Based on an intimate host/virus relationship at each step the viral life cycle is susceptible to inhibiting host cell functions. A summary of examples (see section 4.2) will illustrate the mutual relation. With the exception of the lentiviruses productive infection of target cells by most retroviruses is dependent upon proliferation and concomitant nuclear membrane dissolution of the infected cell. Lentiviruses such as HIV can infect nonproliferating cell types such as macrophages and other terminally differentiated cells overcoming the need for cell division. Activated and resting CD4-positive T helper cells as well as macrophages represent the major target cells for HIV. The role of dendritic cells as well as glia cells in HIV propagation and (neuro)-pathogenesis is discussed controversially.

HIV has been shown to be the etiologic agent of the acquired immunodeficiency syndrome (AIDS). The virus is transmitted by exposure to body fluids of an infected person. Sexual transmission, blood transfusions as well as intravenous drug abuse comprise the major routes. Infection with HIV is characterized by relentless and progressive decline in both number and function of CD4-positive T helper lymphocytes, which play a central role in coordinating immune responses. Ultimately, the weakened immune system is unable to control and eradicate the virus, AIDS develops, which is often accompanied with other opportunistic infections. In the four decades that HIV has afflicted the human population virus spread led to the death of over 22 Million people. It is estimated that about 36 million people worldwide are infected with HIV.

Antiretroviral drug therapy mainly encompassing different combinations of nucleosidic, non-nucleosidic inhibitors of the viral reverse transcriptase as well as protease inhibitors has dramatically improved the lives of those who receive drug treatment. However, current therapies only delay progression of illness and are unable to eradicate the virus. Moreover, drug resistance reappears as a significant problem, close to 50% of the patients fail to efficiently suppress viral replication on treatment mainly due to resistance issues and tolerability/compliance of current drug regimens. Thus, additional HIV therapies are urgently required.

### DETAILED DESCRIPTION OF THE INVENTION

The present invention provides methods and compositions for the diagnosis and treatment of AIDS and HIV-related disorders.

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes and antisense oligonucleotides. Representative molecules are described herein.

The present invention is based, at least in part, on the discovery that nucleic acid and protein molecules, (described infra), are differentially expressed in disease states relative to their expression in normal, or non-disease states. The modulators of the molecules of the present invention, identified according to the methods of the invention can be used to modulate (e.g., inhibit, treat, or prevent) or diagnose a disease, including, but not limited to, AIDS and HIV-related disorders.

“Differential expression”, as used herein, includes both quantitative as well as qualitative differences in the temporal and/or tissue expression pattern of a gene. Thus, a differentially expressed gene may have its expression activated or inactivated in normal versus disease conditions. The degree to which expression differs in normal versus disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic a disease, e.g., AIDS and HIV-related disorders, evaluation, or may be used in methods for identifying compounds useful for the treatment of a disease, e.g., AIDS and HIV-related disorders. In addition, a differentially expressed gene involved in a disease may represent a target gene such that modulation of the level of target gene expression or of target gene product activity will act to cure, heal, alleviate, relieve, alter, remedy, ameliorate, improve or affect a disease condition, e.g., AIDS and HIV-related disorders. Compounds that modulate target gene expression or activity of the target gene product can be used in the treatment of a disease. Although the genes described herein may be differentially expressed with respect to a disease, and/or their products may interact with gene products important to a disease, the genes may also be involved in mechanisms important to additional disease cell processes.

An “AIDS- or HIV-related cell”, as used herein, includes, but is not limited to, thymocytes, dendritic cells, T cells, macrophages, peripheral blood mononuclear cells (PBMC), lymphocytes, monocytes, leukocytes and lymphoid cells.

Molecules of the Present Invention

Gene ID 9145

The human 9145 sequence, known also as 11β-hydroxysteroid dehydrogenase (11β-HSD), is approximately 1348 nucleotides long including untranslated regions (SEQ ID NO:1). The coding sequence, located at about nucleic acid 102 to 980 of SEQ ID NO:1, encodes a 292 amino acid protein (SEQ ID NO:2).

As assessed by TaqMan analysis, 9145 mRNA expression was detected in thymocytes, dendritic cells, dendritic cell CD4+ T cell (DC/CD4) cocultures, T cells and macrophages. 9145 mRNA expression was highly induced by HIV infection in dendritic cells, DC/CD4, macrophages and thymocytes, and CD4+ T cells.

9145 catalyzes the conversion of inactive cortisone to the active glucocorticoid cortisol. The principal glucocorticoid is cortisol. Cortisol is known to have a number of immunosuppressive effects including inhibition of mediators of inflammation, such as cytokines and prostaglandins. Cortisol inhibits production of IL-1 and IL-6 from macrophages and the production of inflammatory effects of bradykinin, platelet-activating factor and serotonin. Cortisol levels are elevated in HIV infected individuals which are correlated with disease progression. HIV patients have been demonstrated to have increased sensitivity to glucocorticoids due to enhanced receptor expression (The Journal of Immunology, 2002, 169: 6361-6368). 9145 mRNA expression is primarily restricted to T cells, dendritic cells, macrophages and liver which contain large numbers of monocyte derived Kupfer cells. 9145 is induced to very high levels of expression following T cell and macrophage activation and following infection with HIV. The induction of 9145 may lead to increased cortisol levels locally and perhaps systemically which could lead to reduced immune responses including the production of proinflammatory cytokines, cytotoxic T cell and NK cell killing of virus-infected cells and enhanced viral replication. Therefore, inhibition of 9145 may decrease glucocorticoid levels, inhibit HIV replication and prevent the immunosuppressive effects of cortisol.

Due to 9145 mRNA expression in thymocytes, dendritic cells, dendritic cell CD4+ T cell (DC/CD4) cocultures, T cells and macrophages, along with its functional role, modulators of 9145 activity would be useful in treating AIDS and HIV-related disorders. 9145 polypeptides of the present invention are useful to screen for modulators of 9145 activity.

Gene ID 1725

The human 1725 sequence (SEQ ID NO:3), known also as angiotensin-converting enzyme, testis-specific isoform (ACE-T), is approximately 2478 nucleotides long including untranslated regions. The coding sequence, located at about nucleic acid 29 to 2227 of SEQ ID NO:3, encodes a 732 amino acid protein (SEQ ID NO:4).

As assessed by TaqMan analysis, 1725 mRNA was highly expressed in macrophages, PBMC, tonsil and lymph node. 1725 mRNA was induced by HIV infection of CD4+ T cells, thymocytes, dendritic cells, dendritic cell/CD4+ T co-cultures and was highly expressed in the permissive Jurkat T cell clone 10H.

1725 is a protease that is expressed at high levels in lymphocytes, dendritic cells and macrophages. 1725 is induced in macrophages by CD4+ T cells (Clin Exp Immunol, 1992, 88(2):288-94) and is known to be involved in activation of CD4+ T cells. 1725 also has high levels of expression in lymphocytes, dendritic cells and macrophages when induced by HIV infection. 1725 is involved in T cell activation required for HIV replication. Therefore, antagonizing 1725 would inhibit HIV replication.

Due to 1725 mRNA expression in macrophages, PBMC, tonsil and lymph node, along with its functional role, modulators of 1725 activity would be useful in treating AIDS and HIV-related disorders. 1725 polypeptides of the present invention are useful to screen for modulators of 1725 activity.

Gene ID 311

The human 311 sequence (SEQ ID NO:5), known also as the nicotinic acid receptor (HM74A), is approximately 2051 nucleotides long including untranslated regions. The coding sequence, located at about nucleic acid 61 to 1224 of SEQ ID NO:5, encodes a 387 amino acid protein (SEQ ID NO:6).

As assessed by TaqMan analysis, 311 mRNA was highly expressed in spleen, tonsil, lymph node and PBMC. 311 mRNA was induced by HIV infection of dendritic cells, dendritic cell/T cell co-cultures and macrophages.

311 is the nicotinic acid receptor, HM74A (JBC, 2003, 278:9869-9874). Administration of nicotinic acid is used in the treatment of dyslipidemia which is believed to inhibit adipocyte lipolysis via the activation of a Gi-coupled receptor. Gi-coupled receptor stimulation results in the activation of MAP and JNK kinases which are involved in the production of cytokines and cell division. HIV replication requires T cell activation. Antagonizing 311 would result in decreased T cell activation and viral replication.

Due to 311 mRNA expression in the spleen, tonsil, lymph node and PBMC, along with its functional role, modulators of 311 activity would be useful in treating AIDS and HIV-related disorders. 311 polypeptides of the present invention are useful to screen for modulators of 311 activity.

Gene ID 837

The human 837 sequence (SEQ ID NO:7), known also as the alpha 7 subunit of the acetylcholine receptor, is approximately 2087 nucleotides long including untranslated regions. The coding sequence, located at about nucleic acid 104 to 1612 of SEQ ID NO:7, encodes a 502 amino acid protein (SEQ ID NO:8).

As assessed by TaqMan analysis, 837 mRNA was highly expressed in peripheral blood lymphocytes (PBL) and tonsil. 837 mRNA was induced by HIV infection of CD4+ T cells, thymocytes, dendritic cells, dendritic cell/CD4+ T co-cultures.

837 is required for an anti-inflammatory response that inhibits TNFα secretion by macrophages (Nature, May 2000, 405(6785):458-462). 837 knockouts display elevated levels of TNFα. TNFα is secreted by macrophages, which enhances HIV replication in a paracrine fashion. Agonizing 837 will reduce the level of TNFα secreted by macrophages, resulting in reduced HIV replication.

Due to 837 mRNA expression in the PBL and tonsil, along with its functional role, modulators of 837 activity would be useful in treating AIDS and HIV-related disorders. 837 polypeptides of the present invention are useful to screen for modulators of 837 activity.

Gene ID 58305

The human 58305 sequence (SEQ ID NO:9), known also as vesicular inhibitory amino acid transporter (GABA and glycine) (hVIAAT), is approximately 2585 nucleotides long including untranslated regions. The coding sequence, located at about nucleic acid 248 to 1825 of SEQ ID NO:9, encodes a 525 amino acid protein (SEQ ID NO:10).

As assessed by transcriptional profiling, 58305 mRNA expression was up regulated in PBMCs of SIV infected rhesus macaque monkeys. This was confirmed by RT-PCR.

T lymphocyte activation is required for viral replication. Activated T cells are highly metabolically active and undergo multiple rounds of cell division. Each cell division requires a doubling of cellular proteins, requiring increased cellular uptake of amino acids for protein synthesis. During HIV replication up to thirty percent of total cellular mRNA can be viral transcripts resulting in high levels of viral protein synthesis. Therefore, inhibition of 58305 will inhibit T cell activation and viral protein synthesis resulting in decreased viral replication.

Due to 58305 mRNA expression in HIV-infected T-cells, along with its functional role, modulators of 58305 activity would be useful in treating AIDS and HIV-related disorders. 58305 polypeptides of the present invention are useful to screen for modulators of 58305 activity.

Gene ID 156

The human 156 sequence (SEQ ID NO:11), known also as formyl peptide receptor-like 2 (FPRL2), is approximately 1062 nucleotides long. The coding sequence, located at about nucleic acid 1 to 1062 of SEQ ID NO:11, encodes a 353 amino acid protein (SEQ ID NO:12).

As assessed by transcriptional profiling, 156 mRNA expression was up regulated in dendritic cell/CD4+ T cell cocultures. TaqMan analysis indicated that 156 mRNA was expressed at relatively low levels in most tissues and was expressed at higher levels in dendritic cell/CD4+ T cells and macrophages. 156 mRNA was also up regulated in HIV infected macrophages, primary CD4+ T lymphocytes, thymocytes and T cells.

Immature dendritic cells (iDC) respond chemotactically and by Ca(2+) mobilization to N-formyl-Met-Leu-Phe and a recently identified synthetic peptide Trp-Lys-Tyr-Met-Val-D-Met (WKYMVm; SEQ ID NO:53), whereas mature dendritic cells (mDC) derived from the same donor only respond to WKYMVm. Furthermore, iDC and mDC express FPRL2 mRNA and protein. As mDC do not express any other members of the human FPR subfamily, FPRL2 expressed by DC must be functional and mediate the effect of WKYMVm on DC (J Leukoc Biol, 2002 September;72(3):598-607).

Stimulation of GPCRs, including 156 which is Gi linked, leads to T cell activation and proliferation. HIV replication requires T cell activation. FPRL2, expressed in myeloid DC, maintains its maturation, suggesting that the interaction of FPRL2 and its endogenous ligand(s) may be involved in regulating DC trafficking during antigen uptake and processing in the periphery as well as the T cell-stimulating phase of immune responses. Therefore antagonizing 156 would provide a means to inhibit T cell activation and HIV replication.

Due to 156 mRNA expression in T lymphocytes and T cell lines, along with its functional role, modulators of 156 activity would be useful in treating AIDS and HIV-related disorders. 156 polypeptides of the present invention are useful to screen for modulators of 156 activity.

Gene ID 14175

The human 14175 sequence (SEQ ID NO:13), known also as serine/threonine protein kinase 10 or lymphocyte-oriented kinase (LOK), is approximately 4221 nucleotides long including untranslated regions. The coding sequence, located at about nucleic acid 51 to 2957 of SEQ ID NO:13, encodes a 968 amino acid protein (SEQ ID NO:14).

As assessed by TaqMan analysis, 14175 mRNA expression was detected in lymphocytes of SIV infected rhesus macaques. TaqMan analysis of an organ recital panel indicated 14175 mRNA was highly expressed in PBMCs compared to other organs. TaqMan analysis of an inflammation panel also confirmed expression of 14175 mRNA in lymphocytes and monocytes.

T lymphocyte activation is required for viral replication. A number of kinases are involved in T cell activation following stimulation through the T cell receptor. 14175 is a new and unique member of the STE20 family with serine/threonine kinase activity and its expression is restricted mostly to lymphoid cells (Immunogenetics, 1999 May;49(5):369-75). 14175 is involved in mitogen-activated protein (MAP) kinase cascades, which is induced by viral replication. 14175 gene expression is slightly induced in response to T cell activation and HIV infection, suggesting that 14175 is required for viral replication. Therefore, antagonizing 14175 will inhibit T cell activation and viral replication.

Due to 14175 mRNA expression in lymphocytes and PBMCs, along with its functional role, modulators of 14175 activity would be useful in treating AIDS and HIV-related disorders. 14175 polypeptides of the present invention are useful to screen for modulators of 14175 activity.

Gene ID 50352

The human 50352 sequence (SEQ ID NO:15), known also as a ubiquitin transferase, is approximately 3513 nucleotides long including untranslated regions. The coding sequence, located at about nucleic acid 82 to 3150 of SEQ ID NO:15, encodes a 1022 amino acid protein (SEQ ID NO:16).

As assessed by TaqMan analysis, 50352 mRNA expression was up regulated in HIV infected dendritic cell/CD4+ T cell cocultures. TaqMan analysis of organ recital panels indicated high expression of 50352 mRNA in PBMCs and macrophages. RT-PCR of HIV infected PBMCs, monocytes and the T cell line CEM indicated increased expression of 50352 mRNA.

Ubiquitin transferases catalyze the addition of ubiquitin to cellular proteins resulting in degradation by the proteosome. Many molecules necessary for transcription of the HIV genome require ubiquitin transferases for regulation, including NFkB. T lymphocyte activation also requires ubiquitin transferases for regulation which is required for viral replication. 50352 is required for processing of HIV Gag proteins. Ubiquitination of cellular proteins precedes degradation or processing of proteins by 50352. Therefore, inhibition of 50352 inhibits T cell activation and viral replication.

Due to 50352 mRNA expression in dendritic cell/CD4+ T cell cocultures and T cell lines, along with its functional role, modulators of 50352 activity would be useful in treating AIDS and HIV-related disorders. 50352 polypeptides of the present invention are useful to screen for modulators of 50352 activity.

Gene ID 32678

The human 32678 sequence (SEQ ID NO:17), known also as an acid-sensing channel (ASIC1), is approximately 3923 nucleotides long including untranslated regions. The coding sequence, located at about nucleic acid 230 to 1954 of SEQ ID NO:17, encodes a 574 amino acid protein (SEQ ID NO:18).

As assessed by transcriptional profiling, 32678 mRNA expression was up regulated in HIV infected thymocytes and the T cell line C8166. TaqMan analysis confirmed increased expression of 32678 mRNA in HIV infected primary macrophages at multiple time points. 32678 mRNA expression was dramatically increased at the peak of infection of two T lymphocyte cell lines, H9 and C8166.

32678 or ASICI is an acid-sensing channel that is permeable to calcium and will cause depolarization of the cell membrane. Depolarization of the cell membrane will open voltage sensitive calcium channels (VSCC's) leading to increased accumulation of intracellular calcium. (Nature, 1997 Mar. 13;386(6621):173-7). Calcium is an important intracellular messenger that is released from intracellular storage compartments and from plasma membrane Ca++ channels following the generation of inositol triphosphate (InsP3). InsP3 is involved in signaling through the T cell receptor (TCR)/CD3 complex resulting in T cell activation (Cell, 1989 Oct. 6;59(1): 15-20). T cell activation through the TCR/CD3 complex is required for HIV replication in T lymphocytes. Therefore, antagonizing 32678 may inhibit signaling through the TCR/CD3 complex resulting in decreased T cell activation and HIV replication.

Due to 32678 mRNA expression in HIV-infected T cells, along with its functional role, modulators of 32678 activity would be useful in treating AIDS and HIV-related disorders. 32678 polypeptides of the present invention are useful to screen for modulators of 32678 activity.

Gene ID 5560

The human 5560 sequence (SEQ ID NO:19), known also as aspartyl protease 3, is approximately 1373 nucleotides long including untranslated regions. The coding sequence, located at about nucleic acid 31 to 1373 of SEQ ID NO:19, encodes a 448 amino acid protein (SEQ ID NO:20).

As assessed by transcriptional profiling, 5560 mRNA expression increased in HIV infected thymocytes and primary CD4+ T cells. 5560 mRNA expression was highly restricted to T cells and lymphoid tissue and is further induced upon T cell activation and HIV infection. Increased 5560 mRNA expression in HIV infected thymocytes, CD4+ T cells, dendritic cells, monocytes and the T cell line ACH2 was confirmed by TaqMan analysis.

5560 is an aspartyl protease. 5560 is greatly induced following HIV infection suggesting that this enzyme is required for efficient viral replication. Some cellular proteases are known to cleave the HIV gap-pol protein precursor. Inhibition of 5560 may inhibit HIV replication.

Due to 5560 mRNA expression in HIV-infected thymocytes and T cells, along with its functional role, modulators of 5560 activity would be useful in treating AIDS and HIV-related disorders. 5560 polypeptides of the present invention are useful to screen for modulators of 5560 activity.

Gene ID 7240

The human 7240 sequence (SEQ ID NO:21), known also as aldehyde dehydrogenase 1 (ALDH1) or retinal dehydrogenase 1, is approximately 1506 nucleotides long. The coding sequence, located at about nucleic acid 1 to 1506 of SEQ ID NO:21, encodes a 501 amino acid protein (SEQ ID NO:22).

As assessed by transcriptional profiling, 7240 mRNA expression was up regulated in HIV infected dendritic cell/CD4+ T cell cocultures (DC/TC). RT-PCR confirmed expression in HIV infected DC/TC, macrophages and the T cell line ACH2.

Metabolism of retinaldehyde to retinoic acid (RA) is tissue-restricted and ALDH1 is expressed at high levels in DC/TC, macrophages and the T cell line ACH2. ALDH1 is required to metabolize retinol to RA to initiate retinoid signaling. (Chem Biol Interact, 2003 Feb. 1;143-144:201-10). Retinoic acid receptor alpha (RXRα) stimulates transcription from the HIV LTR by binding to the nuclear receptor-responsive element in the presence of RA. Inhibitors of ALDH1 would prevent the conversion of retinaldehyde to RA and prevent RXRα stimulated transcription from the HIV LTR resulting in decreased HIV replication. An FDA approved small molecule inhibitor of ALDH1 exists (disulfiram). (J. Biol. Chem., February 1994; 269:5944-5951).

Due to 7240 mRNA expression in HIV infected DC/TC, along with its functional role, modulators of 7240 activity would be useful in treating AIDS and HIV-related disorders. 7240 polypeptides of the present invention are useful to screen for modulators of 7240 activity.

Gene ID 8865

The human 8865 sequence (SEQ ID NO:23), known also as transglutaminase 2 (TGase), is approximately 3257 nucleotides long including untranslated regions. The coding sequence, located at about nucleic acid 136 to 2199 of SEQ ID NO:23, encodes a 687 amino acid protein (SEQ ID NO:24).

As assessed by transcriptional profiling, 8865 mRNA expression was deregulated in gene arrays of HIV infection of dendritic cell/T cell cocultures (DC/TC). RT-PCR confirms expression of 8865 mRNA in HIV infected T cell lines, macrophages, primary CD4+cells and DC/TC.

TGase cross links polyamines to target proteins, and is regulated by the GTP binding activity of TGase. (J. Biol. Chem., Jan. 3, 2003, 278(1):391-399). The transamidation reaction of TGase has been implicated in a number of biological processes including cellular differentiation, and apoptosis. Mitogens, tumor promoters, and cell differentiation inducing agents trigger an intracellular signaling cascade, which involves Ras and Rho GTPases and leads to activation of mitogen-activated protein (MAP) kinases. HIV is also known to induce cellular signaling pathways including MAP kinases. Retinoic acid (RA) promotes activation of TGase and in vivo transamidation of RhoA. RhoA binds/activates RhoA-associated kinase-2 (ROCK-2), a downstream target and an effector of GTP-bound RhoA which promotes activation of MAP/ERK kinases leading to regulation of nuclear events. Inhibition of TGase would prevent the Rho GTPase activation of MAP/ERK kinases resulting in decreased HIV replication.

Due to 8865 mRNA expression in HIV infected T cell lines, macrophages, primary CD4+cells and DC/TC, along with its functional role, modulators of 8865 activity would be useful in treating AIDS and HIV-related disorders. 8865 polypeptides of the present invention are useful to screen for modulators of 8865 activity.

Gene ID 12396

The human 12396 sequence (SEQ ID NO:25), known also as the GPCR GPR41, is approximately 1061 nucleotides long including untranslated regions. The coding sequence, located at about nucleic acid 11 to 1051 of SEQ ID NO:25, encodes a 346 amino acid protein (SEQ ID NO:26).

As assessed by transcriptional profiling, 12396 mRNA expression was restricted primarily to leukocytes. RT-PCR indicated increased 12396 mRNA expression in HIV infected macrophages, dendritic cells, the T cell line ACH2 and in T cells stimulated with antibodies to CD3.

Propionate was the most potent agonist for GPR41. The receptor is coupled to Inositol 1, 4,5-trisphosphate (IP3) formation, intracellular Ca2+ release, ERK1/2 activation and inhibition of cyclic adenosine monophosphate (cAMP) accumulation. GPR41 is coupled exclusively though the Pertussis toxin-sensitive Gi/o family (J. Biol. Chem. 2003 Apr 23; [epub ahead of print]). HIV replication requires T cell activation which requires cAMP accumulation. Agonizing this receptor would result in the inhibition of cAMP accumulation and decreased HIV replication which would be beneficial in the treatment of HIV infection.

Due to 12396 mRNA expression in HIV infected macrophages, dendritic cells, the T cell line ACH2 and in T cells stimulated with antibodies to CD3, along with its functional role, modulators of 12396 activity would be useful in treating AIDS and HIV-related disorders. 12396 polypeptides of the present invention are useful to screen for modulators of 12396 activity.

Gene ID 12397

The human 12397 sequence (SEQ ID NO:27), known also as GPCR GPR43, is approximately 1013 nucleotides long including untranslated regions. The coding sequence, located at about nucleic acid 11 to 1003 of SEQ ID NO:27, encodes a 330 amino acid protein (SEQ ID NO:28).

As assessed by transcriptional profiling, 12397 mRNA expression was restricted primarily to leukocytes. RT-PCR indicated increased 12397 mRNA expression in HIV infected macrophages, dendritic cells and the T cell line ACH2.

Propionate was the most potent agonist for GPR43. The receptor is coupled to IP3 formation, intracellular Ca2+ release, ERK1/2 activation and increased cAMP accumulation. GPR43 displayed a dual coupling through Gi/o and Pertussis toxin-insensitive Gq protein families. GPCR 43 is believed to be involved in the induction of proinflammatory immune responses. (J. Biol. Chem. 2003 Apr 23; [epub ahead of print]). HIV infection is characterized by high level immune activation which correlates with disease progression. Therapies targeting immune activation have shown efficacy in the treatment of HIV in humans. Antagonizing this receptor would prevent activation of the immune response by leukocytes and inhibit HIV infection.

Due to 12397 mRNA expression in HIV infected macrophages, dendritic cells and the T cell line ACH2, along with its functional role, modulators of 12397 activity would be useful in treating AIDS and HIV-related disorders. 12397 polypeptides of the present invention are useful to screen for modulators of 12397 activity.

Gene ID 13644

The human 13644 sequence (SEQ ID NO:29), known also as monocarboxylate transporter 4 (MCT 4), is approximately 1982 nucleotides long including untranslated regions. The coding sequence, located at about nucleic acid 63 to 1460 of SEQ ID NO:29, encodes a 465 amino acid protein (SEQ ID NO:30).

As assessed by transcriptional profiling, 13644 mRNA was highly expressed in T cells and macrophages. 13644 mRNA was expressed at higher levels in HIV permissive macrophages when compared to nonpermissive macrophages. HIV infection induced 13644 mRNA expression in macrophages, dendritic cells (DC) and dendritic cell/T cell cocultures (DC/TC).

MCT4 is most evident in white muscle and other cells with a high glycolytic rate, such as tumor cells and white blood cells, suggesting it is expressed where lactic acid efflux predominates (Biochem. J., 1999, 343:281-299). T cell activation is required for efficient HIV replication. Activated T cells generate the majority of energy needs via glycolysis. Inhibition of MCT4 would lower the level of T cell activation by inhibition of lactic acid efflux, resulting in inhibition of HIV replication (Biochem. J., 1999, 343:281-299).

Due to 13644 mRNA expression in HIV infected macrophages, dendritic cells and T cells, along with its functional role, modulators of 13644 activity would be useful in treating AIDS and HIV-related disorders. 13644 polypeptides of the present invention are useful to screen for modulators of 13644 activity.

Gene ID 19938

The human 19938 sequence (SEQ ID NO:31), known also as kynurenine 3-hydroxylase, is approximately 1999 nucleotides long including untranslated regions. The coding sequence, located at about nucleic acid 53 to 1513 of SEQ ID NO:31, encodes a 486 amino acid protein (SEQ ID NO:32).

As assessed by transcriptional profiling, 19938 mRNA expression was up regulated in HIV infected dendritic cell/CD4+ cell co-cultures (DC/TC). RT-PCR analysis confirmed expression in DC, DC/TC and monocytes.

IDO (Indeolamine 2, 3-dioxygenase) catalyzes the degradation of tryptophan to kynurenine and subsequent catabolic byproducts. IDO is implicated in the induction of T cell tolerance. Local decreases in availability of tryptophan and the presence of its catabolic byproducts inhibit T cell proliferation and may induce apoptotic death (Nature Immunology, 2002, 3:1056-1057). 19938 converts kynurenine to hydroxykynurenine. 19938 mRNA expression is induced in HIV infected DC and DC/TC.

The induction of this enzyme may have a direct effect of HIV infection. T cell tolerance is seen in patients with HIV infection. Increased levels of apoptosis are also seen in human and primate models of pathogenic HIV and SIV infection. The reduction in tryptophan levels and the accumulation of kynurenine may be responsible for increased T cell tolerance as well as increased levels of apoptosis in HIV infected and noninfected T lymphocytes. Therefore, inhibition of 19938 may prevent T cell tolerance and the increased apoptosis of T lymphocytes seen in patients with HIV infection.

Due to 19938 mRNA expression in HIV infected dendritic cell/CD4+ cell co-cultures (DC/TC), along with its functional role, modulators of 19938 activity would be useful in treating AIDS and HIV-related disorders. 19938 polypeptides of the present invention are useful to screen for modulators of 19938 activity.

Gene ID 2077

The human 2077 sequence (SEQ ID NO:33), known also as glomerular epithelial protein 1 (GLEPP1) or protein-tyrosine-phosphatase receptor type 0 precursor, is approximately 5415 nucleotides long including untranslated regions. The coding sequence, located at about nucleic acid 173 to 3739 of SEQ ID NO:33, encodes an 1188 amino acid protein (SEQ ID NO:34).

As assessed by transcriptional profiling, 2077 mRNA was highly expressed in dendritic cells and macrophages. HIV infection induced 2077 mRNA expression in macrophages, dendritic cells (DC) and dendritic cell/T cell cocultures (DC/TC).

2077 is induced by HIV infection suggesting a role in viral replication. 2077 may dephosphorylate cellular proteins involved in inhibition of replication. If this phosphatase enhances viral replication, then antagonizing 2077 would inhibit HIV replication.

Due to 2077 mRNA expression in HIV infected macrophages, dendritic cells (DC) and dendritic cell/T cell cocultures (DC/TC), along with its functional role, modulators of 2077 activity would be useful in treating AIDS and HIV-related disorders. 2077 polypeptides of the present invention are useful to screen for modulators of 2077 activity.

Gene ID 1735

The human 1735 sequence (SEQ ID NO:35), known also as matrix metalloproteinase-9 (MMP-9), is approximately 2373 nucleotides long including untranslated regions. The coding sequence, located at about nucleic acid 6 to 2129 of SEQ ID NO:35, encodes a 707 amino acid protein (SEQ ID NO:36).

As assessed by transcriptional profiling, 1735 mRNA expression increased in HIV infected macrophages and dendritic cells. 1735 mRNA expression was highly restricted to macrophages and dendritic cells and was further induced upon HIV infection. Increased 1735 mRNA expression in HIV infected macrophages and in dendritic cells was confirmed by TaqMan analysis.

1735 is a matrix metalloprotease. 1735 is greatly induced following HIV infection suggesting that this enzyme is required for efficient viral replication. MMP9 is associated with immune activation and may affect leukocyte entry into the brain. (J. Virol. 2001 July; 75(14):6572-83). Inhibition of MMP-9 may result in decreased viral replication as well as decreased HIV associated neuropathy.

Due to 1735 mRNA expression in HIV infected macrophages and dendritic cells, along with its functional role, modulators of 1735 activity would be useful in treating AIDS and HIV-related disorders. 1735 polypeptides of the present invention are useful to screen for modulators of 1735 activity.

Gene ID 1786

The human 1786 sequence (SEQ ID NO:37), known also as granzyme B, is approximately 934 nucleotides long including untranslated regions. The coding sequence, located at about nucleic acid 8 to 751 of SEQ ID NO:37, encodes a 247 amino acid protein (SEQ ID NO:38).

As assessed by transcriptional profiling, 1786 mRNA expression increased in HIV infected thymocytes and primary CD4+ T cells as well as in a Jurkat T cell clone highly permissive to infection. 1786 mRNA expression was highly restricted to T cells and lymphoid tissue and was further induced upon T cell activation and HIV infection. Increased 1786 mRNA expression in HIV infected thymocytes, CD4+ T cells, and in the Jurkat T cell line was confirmed by TaqMan analysis.

Granzyme B is a T cell- and natural killer cell-specific trypsin-like serine protease that is released from effector cells during cytotoxic cell killing. Granzyme B is essential for the induction of DNA fragmentation and apoptosis in target cells. Granzyme B is found in the blood of normal individuals and at increased levels in patients with rheumatoid arthritis and acute EBV and HIV infection suggesting that granzymes have additional biological effects. Granzyme B is known to induce 1L-6 and IL-8 production in fibroblasts and stimulates IL-6, IL-8 and TNF-alpha from monocytes. (J. Immunol. 1998 Apr. 1; 160(7):3610-3616). Proinflamatory cytokines contribute to increased levels of immune activation and viral replication, therefore inhibition of Granzyme B should inhibit HIV replication.

Due to 1786 mRNA expression in HIV infected thymocytes, CD4+ T cells, and in the Jurkat T cell line, along with its functional role, modulators of 1786 activity would be useful in treating AIDS and HIV-related disorders. 1786 polypeptides of the present invention are useful to screen for modulators of 1786 activity.

Gene ID 10220

The human 10220 sequence (SEQ ID NO:39), known also as ionotropic purinergic receptor P2×7, is approximately 1853 nucleotides long including untranslated regions. The coding sequence, located at about nucleic acid 27 to 1814 of SEQ ID NO:39, encodes a 595 amino acid protein (SEQ ID NO:40).

10220 was identified in a transcriptional profile of HIV infected monocytes. It was expressed in most tissues and was expressed at higher levels in monocytes, dendritic cells, thymocytes, T lymphocytes and macrophages. 10220 mRNA expression was up regulated following HIV infection in macrophages as confirmed by RT-PCR.

P2X7 is a human purinoceptor 7 (ATP receptor) which facilitates cation channel activation and secretion of IL-1beta from LPS-primed macrophages (J. Immunol. 2003 Jun. 1; 170(11):5728-38). HIV replication in macrophages stimulates IL-1 production in vivo and in vitro which acts in an autocrine and paracrine manner to enhance HIV replication in T cells and monocytes. Antibodies to IL-1 inhibit the enhanced HIV replication due to IL-1 production in vitro. P2X7 knockout mice are healthy and fertile. Absence of the P2X7R thus leads to an inability of peritoneal macrophages to release IL-1 in response to ATP. (J. Biol. Chem., Jan. 5, 2001; 276(1):125-132). Inhibition of P2X7 dependent IL-1 production would decrease HIV replication and would not have deleterious effects as demonstrated by the knockout mice.

Due to 10220 mRNA expression in HIV infected monocytes and macrophages, along with its functional role, modulators of 10220 activity would be useful in treating AIDS and HIV-related disorders. 10220 polypeptides of the present invention are useful to screen for modulators of 10220 activity.

Gene ID 17822

The human 17822 sequence (SEQ ID NO:41), a dipeptidase similar to microsomal dipeptidase precursor 1 (MDP1), is approximately 1700 nucleotides long including untranslated regions. The coding sequence, located at about nucleic acid 125 to1585 of SEQ ID NO:41, encodes a 486 amino acid protein (SEQ ID NO:42).

As assessed by TaqMan analysis, 17822 mRNA was highly expressed in dendritic cells and macrophages. 17822 mRNA expression was induced to higher levels in HIV permissive vs. nonpermissive macrophages. HIV infection induced 17822 mRNA expression in macrophages, dendritic cells (DC) and dendritic cell/T cell cocultures (DC/TC).

17822 is a membrane-bound dipeptidase. 17822 catalyzes the conversion of leukotriene D4 (LTD4) to leukotriene E4 (LTE4). (The FASEB Journal. 2003; 17:1313-1315). 17822 may also participate in immune/inflammatory processes involving leukotrienes which are elevated in patients with asthma and may play a role in pathogenesis. Proinflammatory molecules enhance HIV infection and it is possible that LTE4 may enhance replication, therefore inhibition of 17822 should inhibit viral replication.

Due to 17822 mRNA expression in HIV infected macrophages, dendritic cells (DC) and dendritic cell/T cell cocultures (DC/TC), along with its functional role, modulators of 17822 activity would be useful in treating AIDS and HIV-related disorders. 17822 polypeptides of the present invention are useful to screen for modulators of 17822 activity.

Gene ID 33945

The human 33945 sequence (SEQ ID NO:43), known as UDP-N-acetyl-alpha-D-galactosamine:polypeptide N-acetylgalactosaminyltransferase 12 (GalNAc-T12), is approximately 2850 nucleotides long including untranslated regions. The coding sequence, located at about nucleic acid 81 to 1826 of SEQ ID NO:43, encodes a 581 amino acid protein (SEQ ID NO:44).

33945 was identified by transcriptional profiling of HIV infected monocytes. 33945 mRNA was highly expressed in T cells, dendritic cells and macrophages and was further induced upon HIV infection of monocytes, as confirmed by TaqMan analysis.

GalNAc-T12 is involved in O-linked glycosylation of many substrates including the V3 loop of HIV envelope. (FEBS Lett., 2002 Jul. 31; 524(1-3):211-8). 33945 is greatly induced following HIV infection suggesting that this enzyme is required for efficient viral replication. Glycosylation of the HIV envelope is required for infectivity. Inhibitors of 33945 would inhibit the infectivity of HIV by preventing O-linked glycosylation of the V3 loop of HIV envelope.

Due to 33945 mRNA expression in HIV infected monocytes, along with its functional role, modulators of 33945 activity would be useful in treating AIDS and HIV-related disorders. 33945 polypeptides of the present invention are useful to screen for modulators of 33945 activity.

Gene ID 43748

The human 43748 sequence (SEQ ID NO:45), known also as aquaporin 9 (AQP9), is approximately 2890 nucleotides long including untranslated regions. The coding sequence, located at about nucleic acid 229 to 1116 of SEQ ID NO:45, encodes a 295 amino acid protein (SEQ ID NO:46).

As assessed by TaqMan analysis, 43748 mRNA was highly expressed in dendritic cells and macrophages. 43748 mRNA expression was increased in HIV permissive vs. nonpermissive macrophages. HIV infection induced 43748 mRNA expression in macrophages, dendritic cells (DC) and dendritic cell/T cell cocultures (DC/TC).

AQP9 is most evident in white muscle and other cells with a high glycolytic rate, such as tumor cells and white blood cells, suggesting it is expressed where lactic acid efflux predominates. T cell activation is required for efficient HIV replication. Activated T cells generate the majority of their energy needs via glycolysis. AQP9 is most predominant in white blood cells and it transports glycerol and urea at physiological pH. (Proc Natl Acad Sci USA, 2003 Mar. 4; 100(5):2945-50.; E pub 2003 Feb 19). Inhibition of AQP9 would lower the level of T cell metabolic activity by inhibition of transport of glycerol and urea, resulting in inhibition of HIV replication.

Due to 43748 mRNA expression in HIV infected macrophages, dendritic cells (DC) and dendritic cell/T cell cocultures (DC/TC), along with its functional role, modulators of 43748 activity would be useful in treating AIDS and HIV-related disorders. 43748 polypeptides of the present invention are useful to screen for modulators of 43748 activity.

Gene ID 47161

The human 47161 sequence (SEQ ID NO:47), known also as N-acetylgalactosaminyltransferase 6 (GalNAc-T6), is approximately 1869 nucleotides long. The coding sequence, located at about nucleic acid 1 to 1869 of SEQ ID NO:47, encodes a 622 amino acid protein (SEQ ID NO:48).

47161 was identified by transcriptional profiling of HIV infected monocytes. 47161 mRNA expression was very restricted and was highly expressed in T cells, dendritic cells and macrophages and is further induced upon HIV infection of monocytes, as confirmed by TaqMan analysis.

GalNAc-T6 is involved in O-linked glycosylation of many substrates including the V3 loop of HIV envelope. (J Biol. Chem., 1999 Sep. 3; 274(36):25362-70). 47161 is greatly induced following HIV infection suggesting that this enzyme is required for efficient viral replication. Glycosylation of the HIV envelope is required for infectivity. Inhibitors of 47161 would inhibit the infectivity of HIV by preventing O-linked glycosylation of the V3 loop of HIV envelope.

Due to 47161 mRNA expression in HIV infected monocytes, along with its functional role, modulators of 47161 activity would be useful in treating AIDS and HIV-related disorders. 47161 polypeptides of the present invention are useful to screen for modulators of 47161 activity.

Gene ID 81982

The human 81982 sequence (SEQ ID NO:49), known also as “probable serine protease HTRA4 precursor”, is approximately 1544 nucleotides long including untranslated regions. The coding sequence, located at about nucleic acid 87 to 1517 of SEQ ID NO:49, encodes a 476 amino acid protein (SEQ ID NO:50).

As assessed by transcriptional profiling, 81982 mRNA expression was increased in HIV infected macrophages and dendritic cells. 81982 mRNA expression was highly restricted to macrophages and dendritic cells and was further induced upon HIV infection. Increased 81982 mRNA expression in HIV infected macrophages and dendritic cells was confirmed by TaqMan analysis.

81982 is a serine protease. 81982 is greatly induced following HIV infection suggesting that this enzyme is required for efficient viral replication. 5-hydroxytryptamine (serotonin) receptor 4 (HTr4) is in the secretory pathway and is involved in degrading damaged proteins and signal peptides found on HIV proteins. High level protein synthesis occurs during HIV replication. These proteins accumulate inside the cell membrane and assemble to form virions. In vivo, aggregate formation is a highly favorable process due to the extremely high intracellular protein concentrations. HTr4 is a protease-chaperone heat shock protein that prevents aggregate formation (Molecular Cell, Sept. 2002; 10:443-455). Inhibition of this enzyme may interfere with virion protein assembly, maturation and release of viral particles.

Due to 81982 mRNA expression in HIV infected macrophages and dendritic cells, along with its functional role, modulators of 81982 activity would be useful in treating AIDS and HIV-related disorders. 81982 polypeptides of the present invention are useful to screen for modulators of 81982 activity.

Gene ID 46777

The human 46777 sequence (SEQ ID NO:51), known also as disintegrin-protease, is approximately 2187 nucleotides long including untranslated regions. The coding sequence, located at about nucleic acid 61 to 1473 of SEQ ID NO:51, encodes a 470 amino acid protein (SEQ ID NO:52).

As assessed by transcriptional profiling, 46777 mRNA expression levels increased in HIV infected thymocytes and primary DC/TC (dendritic cell/T cell cocultures) compared to non-infected samples. 46777 tissue expression was highly restricted to T cells and lymphoid tissue and was further induced upon T cell activation and HIV infection. TaqMan experiments additionally confirmed that 46777 mRNA expression was increased in HIV infected thymocytes, dendritic cells, and monocytes.

46777 is a protease which is greatly induced following HIV infection, suggesting that this enzyme is required for efficient viral replication. Some cellular proteases are known to cleave the HIV gap-pol protein precursor. Therefore, inhibition of 46777 should inhibit HIV replication.

Therefore, based on the specific expression and regulation of 46777 in HIV infected tissues and cell types, such thymocytes and T cells, modulators of 46777 activity would be useful in treating AIDS or an HIV-related disorder. 46777 polypeptides of the present invention would be useful in screening for modulators of 46777 activity.

Various aspects of the invention are described in further detail in the following subsections:

I. Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins, have a stimulatory or inhibitory effect on, for example, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity, or have a stimulatory or inhibitory effect on, for example, the expression or activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate. Compounds identified using the assays described herein may be useful for treating AIDS or an HIV-related disorder.

These assays are designed to identify compounds that bind to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, bind to other intracellular or extracellular proteins that interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, and interfere with the interaction of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein with other intercellular or extracellular proteins. For example, in the case of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, which is a transmembrane receptor-type protein, such techniques can identify ligands for such a receptor. A 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein ligand or substrate can, for example, be used to ameliorate at least one symptom of AIDS or an HIV-related disorder. Such compounds may include, but are not limited to peptides, antibodies, or small organic or inorganic compounds. Such compounds may also include other cellular proteins.

Compounds identified via assays such as those described herein may be useful, for example, for treating AIDS or an HIV-related disorder. In instances whereby AIDS or an HIV-related disorder results from an overall lower level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression and/or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein in a cell or tissue, compounds that interact with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein may include compounds which accentuate or amplify the activity of the bound 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Such compounds would bring about an effective increase in the level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein activity, thus ameliorating symptoms.

In other instances, mutations within the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene may cause aberrant types or excessive amounts of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins to be made which have a deleterious effect that leads to AIDS or an HIV-related disorder. Similarly, physiological conditions may cause an excessive increase in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression leading to AIDS or an HIV-related disorder. In such cases, compounds that bind to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein may be identified that inhibit the activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Assays for testing the effectiveness of compounds identified by techniques such as those described in this section are discussed herein.

In one embodiment, the invention provides assays for screening candidate or test compounds which are substrates of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or polypeptide or biologically active portion thereof. In another embodiment, the invention provides assays for screening candidate or test compounds which bind to or modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof is contacted with a test compound and the ability of the test compound to modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is determined. Determining the ability of the test compound to modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with AIDS or an HIV-related disorder, or the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-regulated transcription factor. The cell can be of mammalian origin, e.g., a neural cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of AIDS or an HIV-related disorder.

The ability of the test compound to modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding to a substrate or to bind to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can also be determined. Determining the ability of the test compound to modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding to a substrate can be accomplished, for example, by coupling the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate with a radioisotope or enzymatic label such that binding of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be determined by detecting the labeled 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate in a complex. 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 could also be coupled with a radioisotope or enzymatic label to monitor the ability of a test compound to modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate in a complex. Determining the ability of the test compound to bind 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be accomplished, for example, by coupling the compound with a radioisotope or enzymatic label such that binding of the compound to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be determined by detecting the labeled 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 compound in a complex. For example, compounds (e.g., 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 ligand or substrate) to interact with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 without the labeling of any of the interactants. For example, a microphysiometer can be used to detect the interaction of a compound with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 without the labeling of either the compound or the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777.

In another embodiment, an assay is a cell-based assay comprising contacting a cell expressing a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate) with a test compound and determining the ability of the test compound to modulate (e.g., stimulate or inhibit) the activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule. Determining the ability of the test compound to modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule can be accomplished, for example, by determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to bind to or interact with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule.

Determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or a biologically active fragment thereof, to bind to or interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule can be accomplished by one of the methods described above for determining direct binding. In a preferred embodiment, determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to bind to or interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof, is contacted with a test compound and the ability of the test compound to bind to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof is determined. Preferred biologically active portions of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins to be used in assays of the present invention include fragments which participate in interactions with non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 molecules, e.g., fragments with high surface probability scores. Binding of the test compound to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be determined either directly or indirectly as described above. In a preferred embodiment, the assay includes contacting the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof with a known compound which binds 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 to form an assay mixture, contacting the assay mixture with a test compound, and determining the ability of the test compound to interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, wherein determining the ability of the test compound to interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein comprises determining the ability of the test compound to preferentially bind to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 or biologically active portion thereof as compared to the known compound. Compounds that modulate the interaction of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 with a known target protein may be useful in regulating the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, especially a mutant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

In another embodiment, the assay is a cell-free assay in which a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof is contacted with a test compound and the ability of the test compound to modulate (e.g., stimulate or inhibit) the activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof is determined. Determining the ability of the test compound to modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be accomplished, for example, by determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to bind to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule by one of the methods described above for determining direct binding. Determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to bind to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be accomplished by determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to further modulate the activity of a downstream effector of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule. For example, the activity of the effector molecule on an appropriate target can be determined or the binding of the effector to an appropriate target can be determined as previously described.

In yet another embodiment, the cell-free assay involves contacting a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or biologically active portion thereof with a known compound which binds the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to form an assay mixture, contacting the assay mixture with a test compound, and determining the ability of the test compound to interact with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, wherein determining the ability of the test compound to interact with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein comprises determining the ability of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to preferentially bind to or modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule.

In more than one embodiment of the above assay methods of the present invention, it may be desirable to immobilize either 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 or its target molecule to facilitate separation of complexed from uncomplexed forms of one or both of the proteins, as well as to accommodate automation of the assay. Binding of a test compound to a 9145, 1725, 311, 837, 58305, 156, 14175, 50352, 32678, 5560, 7240; 8865, 12396, 12397, 13644, 19938, 2077, 1735, 1786, 10220, 17822, 33945, 43748, 47161, 81982 or 46777 protein, or interaction of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding or activity determined using standard techniques.

Other techniques for immobilizing proteins on matrices can also be used in the screening assays of the invention. For example, either a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 target molecule can be immobilized utilizing conjugation of biotin and streptavidin. Biotinylated 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or target molecules but which do not interfere with binding of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to its target molecule can be derivatized to the wells of the plate, and unbound target or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein trapped in the wells by antibody conjugation. Methods for detecting such complexes, in addition to those described above for the GST-immobilized complexes, include immunodetection of complexes using antibodies reactive with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or target molecule, as well as enzyme-linked assays which rely on detecting an enzymatic activity associated with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or target molecule.

In another embodiment, modulators of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression are identified in a method wherein a cell is contacted with a candidate compound and the expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein in the cell is determined. The level of expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein in the presence of the candidate compound is compared to the level of expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein in the absence of the candidate compound. The candidate compound can then be identified as a modulator of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression based on this comparison. For example, when expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein is greater (statistically significantly greater) in the presence of the candidate compound than in its absence, the candidate compound is identified as a stimulator of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein expression. Alternatively, when expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein is less (statistically significantly less) in the presence of the candidate compound than in its absence, the candidate compound is identified as an inhibitor of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein expression. The level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein expression in the cells can be determined by methods described herein for detecting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or protein.

In yet another aspect of the invention, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 (“9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-binding proteins” or “9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-bp”) and are involved in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Such 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-binding proteins are also likely to be involved in the propagation of signals by the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 targets as, for example, downstream elements of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-mediated signaling pathway. Alternatively, such 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-binding proteins are likely to be 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 inhibitors.

The two-hybrid system is based on the modular nature of most transcription factors, which consist of separable DNA-binding and activation domains. Briefly, the assay utilizes two different DNA constructs. In one construct, the gene that codes for a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

In another aspect, the invention pertains to a combination of two or more of the assays described herein. For example, a modulating agent can be identified using a cell-based or a cell free assay, and the ability of the agent to modulate the activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be confirmed in vivo, e.g., in an animal such as an animal model for AIDS or an HIV-related disorder, as described herein.

This invention further pertains to novel agents identified by the above-described screening assays. Accordingly, it is within the scope of this invention to further use an agent identified as described herein in an appropriate animal model. For example, an agent identified as described herein (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulating agent, an antisense 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecule, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-specific antibody, or a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate at least one symptom of AIDS or an HIV-related disorder. Cell-based and animal model-based assays for the identification of compounds exhibiting such an ability to ameliorate at least one symptom of AIDS or an HIV-related disorder are described herein.

In addition, animal-based models of AIDS or an HIV-related disorder, such as those described herein, may be used to identify compounds capable of treating AIDS or an HIV-related disorder. Such animal models may be used as test substrates for the identification of drugs, pharmaceuticals, therapies, and interventions which may be effective in treating AIDS or an HIV-related disorder. For example, animal models may be exposed to a compound, suspected of exhibiting an ability to treat AIDS or an HIV-related disorder, at a sufficient concentration and for a time sufficient to elicit such an amelioration of at least one symptom of AIDS or an HIV-related disorder in the exposed animals. The response of the animals to the exposure may be monitored by assessing the reversal of the symptoms of AIDS or an HIV-related disorder before and after treatment.

With regard to intervention, any treatments which reverse any aspect of a viral disorder (i.e. have an effect on AIDS or an HIV-related disorder) should be considered as candidates for AIDS or an HIV-related disorder therapeutic intervention. Dosages of test agents may be determined by deriving dose-response curves.

Additionally, gene expression patterns may be utilized to assess the ability of a compound to ameliorate at least one symptom of AIDS or an HIV-related disorder. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences may be used as probes and/or PCR primers for the generation and corroboration of such gene expression profiles.

Gene expression profiles may be characterized for known states, either viral disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of AIDS or an HIV-related disorder disease model system to more closely resemble the control system. Administration of a compound may, alternatively, cause the gene expression profile of a control system to begin to mimic AIDS or an HIV-related disorder or AIDS or an HIV-related disease state. Such a compound may, for example, be used in further characterizing the compound of interest, or may be used in the generation of additional animal models.

II. Cell- and Animal-Based Model Systems

Described herein are cell- and animal-based systems which act as models for AIDS or an HIV-related disorder. These systems may be used in a variety of applications. For example, the cell- and animal-based model systems may be used to further characterize differentially expressed genes associated with AIDS or an HIV-related disorder, e.g., 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. In addition, animal- and cell-based assays may be used as part of screening strategies designed to identify compounds which are capable of ameliorating at least one symptom of AIDS or an HIV-related disorder, as described, below. Thus, the animal- and cell-based models may be used to identify drugs, pharmaceuticals, therapies and interventions which may be effective in treating AIDS or an HIV-related disorder. Furthermore, such animal models may be used to determine the LD50 and the ED50 in animal subjects, and such data can be used to determine the in vivo efficacy of potential urological disorder treatments.

A. Animal-Based Systems

Animal-based model systems of urological disorder may include, but are not limited to, non-recombinant and engineered transgenic animals.

Non-recombinant animal models for AIDS or an HIV-related disorder may include, for example, genetic models.

Additionally, animal models exhibiting AIDS or an HIV-related disorder may be engineered by using, for example, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences described above, in conjunction with techniques for producing transgenic animals that are well known to those of skill in the art. For example, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences may be introduced into, and overexpressed in, the genome of the animal of interest, or, if endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences are present, they may either be overexpressed or, alternatively, be disrupted in order to underexpress or inactivate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression.

The host cells of the invention can also be used to produce non-human transgenic animals. For example, in one embodiment, a host cell of the invention is a fertilized oocyte or an embryonic stem cell into which 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-coding sequences have been introduced. Such host cells can then be used to create non-human transgenic animals in which exogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences have been introduced into their genome or homologous recombinant animals in which endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences have been altered. Such animals are useful for studying the function and/or activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 and for identifying and/or evaluating modulators of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-encoding nucleic acid into the male pronuclei of a fertilized oocyte, e.g., by microinjection, retroviral infection, and allowing the oocyte to develop in a pseudopregnant female foster animal. The 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 cDNA sequence can be introduced as a transgene into the genome of a non-human animal. Alternatively, a nonhuman homologue of a human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, such as a mouse or rat 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, can be used as a transgene. Alternatively, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene homologue, such as another 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 family member, can be isolated based on hybridization to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 cDNA sequences and used as a transgene. Intronic sequences and polyadenylation signals can also be included in the transgene to increase the efficiency of expression of the transgene. A tissue-specific regulatory sequence(s) can be operably linked to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 transgene to direct expression of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to particular cells. Methods for generating transgenic animals via embryo manipulation and microinjection, particularly animals such as mice, have become conventional in the art and are described, for example, in U.S. Pat. Nos. 4,736,866 and 4, 870, 009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 transgene in its genome and/or expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA in tissues or cells of the animals. A transgenic founder animal can then be used to breed additional animals carrying the transgene. Moreover, transgenic animals carrying a transgene encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can further be bred to other transgenic animals carrying other transgenes.

To create a homologous recombinant animal, a vector is prepared which contains at least a portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene into which a deletion, addition or substitution has been introduced to thereby alter, e.g., functionally disrupt, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene. The 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene can be a human gene but more preferably, is a non-human homologue of a human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene. For example, a rat 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene can be used to construct a homologous recombination nucleic acid molecule, e.g., a vector, suitable for altering an endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene in the mouse genome. In a preferred embodiment, the homologous recombination nucleic acid molecule is designed such that, upon homologous recombination, the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene is functionally disrupted (i.e., no longer encodes a functional protein; also referred to as a “knock out” vector). Alternatively, the homologous recombination nucleic acid molecule can be designed such that, upon homologous recombination, the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene is mutated or otherwise altered but still encodes functional protein (e.g., the upstream regulatory region can be altered to thereby alter the expression of the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein). In the homologous recombination nucleic acid molecule, the altered portion of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene is flanked at its 5′ and 3′ ends by additional nucleic acid sequence of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene to allow for homologous recombination to occur between the exogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene carried by the homologous recombination nucleic acid molecule and an endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene in a cell, e.g., an embryonic stem cell. The additional flanking 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene has homologously recombined with the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 transgenic animals that express 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 peptide (detected immunocytochemically, using antibodies directed against 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 epitopes) at easily detectable levels should then be further evaluated to identify those animals which display a characteristic HIV-related disorder.

B. Cell-Based Systems

Cells that contain and express 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences which encode a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, and, further, exhibit cellular phenotypes associated AIDS or an HIV-related disorder, may be used to identify compounds that exhibit an effect on AIDS or an HIV-related disorder. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), and T-cell or monocyte cell lines. Further, such cells may include recombinant, transgenic cell lines. For example, the AIDS or an HIV-related disorder animal models of the invention, discussed above, may be used to generate cell lines, containing one or more cell types involved in AIDS or an HIV-related disorder, that can be used as cell culture models for this disorder. While primary cultures derived from the urological disorder model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in AIDS or an HIV-related disorder may be transfected with sequences capable of increasing or decreasing the amount of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression within the cell. For example, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences may be introduced into, and overexpressed in, the genome of the cell of interest, or, if endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences are present, they may be either overexpressed or, alternatively disrupted in order to underexpress or inactivate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression.

In order to overexpress a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, the coding portion of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequence, such a sequence may be isolated and engineered such that when reintroduced into the genome of the cell type of interest, the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 alleles will be inactivated. Preferably, the engineered 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence is introduced via gene targeting such that the endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence is disrupted upon integration of the engineered 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence into the cell's genome. Transfection of host cells with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 genes is discussed, above.

Cells treated with compounds or transfected with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 genes can be examined for phenotypes associated with AIDS or an HIV-related disorder.

Transfection of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid may be accomplished by using standard techniques (described in, for example, Ausubel (1989) supra). Transfected cells should be evaluated for the presence of the recombinant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences, for expression and accumulation of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA, and for the presence of recombinant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein production. In instances wherein a decrease in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression is desired, standard techniques may be used to demonstrate whether a decrease in endogenous 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression and/or in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein production is achieved.

Also provided are cells or a purified preparation thereof, e.g., human cells, in which an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 is under the control of a regulatory sequence that does not normally control the expression of the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene. For example, an endogenous 9118, 990, 17662, 81982, 630, 21472, 17692, 19290, 21620, 21689, 28899, 53659, 64549, 9465, 23544, 7366, 27417, 57259, 21844, 943, 2061, 5891, 9137, 13908, 14310, 17600, 25584, 27824, 28469, 38947, 53003, 965, 56639, 9661, 16052, 1521, 6662, 13913, 12405 or 5014 gene, e.g., a gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

III. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein and/or nucleic acid expression as well as 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity, in the context of a biological sample (e.g., blood, serum, cells, e.g., endothelial cells, or tissue, e.g., vascular tissue, lymphoid tissue, peripheral blood cells) to thereby determine whether an individual is afflicted with a predisposition or is experiencing AIDS or an HIV-related disorder. The invention also provides for prognostic (or predictive) assays for determining whether an individual is at risk of developing AIDS or an HIV-related disorder. For example, mutations in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene can be assayed for in a biological sample. Such assays can be used for prognostic or predictive purpose to thereby phophylactically treat an individual prior to the onset of AIDS or an HIV-related disorder.

Another aspect of the invention pertains to monitoring the influence of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulators (e.g., anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 ribozymes) on the expression or activity of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 in clinical trials.

These and other agents are described in further detail in the following sections.

A. Diagnostic Assays

To determine whether a subject is afflicted with a disease, a biological sample may be obtained from a subject and the biological sample may be contacted with a compound or an agent capable of detecting a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or nucleic acid (e.g., mRNA or genomic DNA) that encodes a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, in the biological sample. A preferred agent for detecting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or genomic DNA is a labeled nucleic acid probe capable of hybridizing to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or genomic DNA. The nucleic acid probe can be, for example, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion thereof, such as an oligonucleotide of at least 15, 20, 25, 30, 25, 40, 45, 50, 100, 250 or 500 nucleotides in length and sufficient to specifically hybridize under stringent conditions to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA or genomic DNA. Other suitable probes for use in the diagnostic assays of the invention are described herein.

A preferred agent for detecting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein in a sample is an antibody capable of binding to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA, protein, or genomic DNA in a biological sample in vitro as well as in vivo. For example, in vitro techniques for detection of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA include Northern hybridizations and in situ hybridizations. In vitro techniques for detection of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein include enzyme linked immunosorbent assays (ELISAs), Western blots, immunoprecipitations and immunofluorescence. In vitro techniques for detection of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 genomic DNA include Southern hybridizations. Furthermore, in vivo techniques for detection of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein include introducing into a subject a labeled anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA, or genomic DNA, such that the presence of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA or genomic DNA is detected in the biological sample, and comparing the presence of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA or genomic DNA in the control sample with the presence of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA or genomic DNA in the test sample.

B. Prognostic Assays

The present invention further pertains to methods for identifying subjects having or at risk of developing a disease associated with aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity.

As used herein, the term “aberrant” includes a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity which deviates from the wild type 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity. Aberrant expression or activity includes increased or decreased expression or activity, as well as expression or activity which does not follow the wild type developmental pattern of expression or the subcellular pattern of expression. For example, aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity is intended to include the cases in which a mutation in the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene causes the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene to be under-expressed or over-expressed and situations in which such mutations result in a non-functional 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or a protein which does not function in a wild-type fashion, e.g., a protein which does not interact with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate, or one which interacts with a non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate.

The assays described herein, such as the preceding diagnostic assays or the following assays, can be used to identify a subject having or at risk of developing a disease. A biological sample may be obtained from a subject and tested for the presence or absence of a genetic alteration. For example, such genetic alterations can be detected by ascertaining the existence of at least one of 1) a deletion of one or more nucleotides from a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 2) an addition of one or more nucleotides to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 3) a substitution of one or more nucleotides of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 4) a chromosomal rearrangement of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 5) an alteration in the level of a messenger RNA transcript of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 6) aberrant modification of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, such as of the methylation pattern of the genomic DNA, 7) the presence of a non-wild type splicing pattern of a messenger RNA transcript of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, 8) a non-wild type level of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-protein, 9) allelic loss of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, and 10) inappropriate post-translational modification of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-protein.

As described herein, there are a large number of assays known in the art which can be used for detecting genetic alterations in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene. For example, a genetic alteration in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene may be detected using a probe/primer in a polymerase chain reaction (PCR) (see, e.g., U.S. Pat. Nos. 4,683,195 and 4, 683, 202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene under conditions such that hybridization and amplification of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene in a biological sample and detect mutations by comparing the sequence of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence, e.g., a wild-type 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator (e.g., an agonist, antagonist, peptidomimetic, protein, peptide, nucleic acid, or small molecule) to effectively treat a disease.

C. Monitoring of Effects During Clinical Trials

The present invention further provides methods for determining the effectiveness of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator identified herein) in treating a disease. For example, the effectiveness of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator in increasing 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression, protein levels, or in upregulating 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity, can be monitored in clinical trials of subjects exhibiting decreased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression, protein levels, or downregulated 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Alternatively, the effectiveness of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator in decreasing 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression, protein levels, or in downregulating 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity, can be monitored in clinical trials of subjects exhibiting increased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression, protein levels, or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. In such clinical trials, the expression or activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, and preferably, other genes that have been implicated in nociception can be used as a “read out” or marker of the phenotype of a particular cell.

For example, and not by way of limitation, genes, including 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777, that are modulated in cells by treatment with an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity (e.g., identified in a screening assay as described herein) can be identified. Thus, to study the effect of agents which modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity on subjects suffering from AIDS or an HIV-related disorder in, for example, a clinical trial, cells can be isolated and RNA prepared and analyzed for the levels of expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 and other genes implicated in the HIV-related disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 or other genes. In this way, the gene expression pattern can serve as a marker, indicative of the physiological response of the cells to the agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. This response state may be determined before, and at various points during treatment of the individual with the agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity.

In a preferred embodiment, the present invention provides a method for monitoring the effectiveness of treatment of a subject with an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA, or genomic DNA in the pre-administration sample; (iii) obtaining one or more post-administration samples from the subject; (iv) detecting the level of expression or activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA, or genomic DNA in the post-administration samples; (v) comparing the level of expression or activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA, or genomic DNA in the pre-administration sample with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, mRNA, or genomic DNA in the post administration sample or samples; and (vi) altering the administration of the agent to the subject accordingly. For example, increased administration of the agent may be desirable to increase the expression or activity of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 to higher levels than detected, i.e., to increase the effectiveness of the agent. Alternatively, decreased administration of the agent may be desirable to decrease expression or activity of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 to lower levels than detected, i.e. to decrease the effectiveness of the agent. According to such an embodiment, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity may be used as an indicator of the effectiveness of an agent, even in the absence of an observable phenotypic response.

IV. Methods of Treatment:

The present invention provides for both prophylactic and therapeutic methods of treating a subject, e.g., a human, at risk of (or susceptible to) a disease. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 molecules of the present invention or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a disease by administering to the subject an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Subjects at risk for AIDS or an HIV-related disorder, e.g., can be identified by, for example, any or a combination of the diagnostic or prognostic assays described herein. Administration of a prophylactic agent can occur prior to the manifestation of symptoms characteristic of aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity, such that a disease is prevented or, alternatively, delayed in its progression. Depending on the type of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 aberrancy, for example, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 agonist or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antagonist agent can be used for treating the subject. The appropriate agent can be determined based on screening assays described herein.

B. Therapeutic Methods

Described herein are methods and compositions whereby AIDS or an HIV-related disorder may be ameliorated. Certain urological disorders are brought about, at least in part, by an excessive level of a gene product, or by the presence of a gene product exhibiting an abnormal or excessive activity. As such, the reduction in the level and/or activity of such gene products would bring about the amelioration of at least one symptom of AIDS or an HIV-related disorder. Techniques for the reduction of gene expression levels or the activity of a protein are discussed below.

Alternatively, certain other HIV-related disorders are brought about, at least in part, by the absence or reduction of the level of gene expression, or a reduction in the level of a protein's activity. As such, an increase in the level of gene expression and/or the activity of such proteins would bring about the amelioration of at least one symptom of AIDS or an HIV-related disorder.

In some cases, the up-regulation of a gene in a disease state reflects a protective role for that gene product in responding to the disease condition. Enhancement of such a gene's expression, or the activity of the gene product, will reinforce the protective effect it exerts. Some urological disease states may result from an abnormally low level of activity of such a protective gene. In these cases also, an increase in the level of gene expression and/or the activity of such gene products would bring about the amelioration of a least one symptom of AIDS or an HIV-related disorder. Techniques for increasing target gene expression levels or target gene product activity levels are discussed herein.

Accordingly, another aspect of the invention pertains to methods of modulating 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity for therapeutic purposes. Accordingly, in an exemplary embodiment, the modulatory method of the invention involves contacting a cell with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 or agent that modulates one or more of the activities of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein activity associated with the cell (e.g., an endothelial cell, ovarian cell, T-cell or monocyte). An agent that modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein activity can be an agent as described herein, such as a nucleic acid or a protein, a naturally-occurring target molecule of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 ligand or substrate), a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 agonist or antagonist, a peptidomimetic of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 agonist or antagonist, or other small molecule. In one embodiment, the agent stimulates one or more 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activities. Examples of such stimulatory agents include active 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein and a nucleic acid molecule encoding 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 that has been introduced into the cell. In another embodiment, the agent inhibits one or more 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activities. Examples of such inhibitory agents include antisense 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules, anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies, and 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or nucleic acid molecule. In one embodiment, the method involves administering an agent (e.g., an agent identified by a screening assay described herein), or combination of agents that modulates (e.g., upregulates or downregulates) 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity. In another embodiment, the method involves administering a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or nucleic acid molecule as therapy to compensate for reduced, aberrant, or unwanted 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity.

Stimulation of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is desirable in situations in which 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 is abnormally downregulated and/or in which increased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is likely to have a beneficial effect. Likewise, inhibition of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is desirable in situations in which 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 is abnormally upregulated and/or in which decreased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is likely to have a beneficial effect.

(i) Methods for Inhibiting Target Gene Expression, Synthesis, or Activity

As discussed above, genes involved in viral disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate at least one symptom of AIDS or an HIV-related disorder. Such molecules may include, but are not limited to, small organic molecules, peptides, antibodies, and the like.

For example, compounds can be administered that compete with endogenous ligand for the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. The resulting reduction in the amount of ligand-bound 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein will modulate endothelial cell physiology. Compounds that can be particularly useful for this purpose include, for example, soluble proteins or peptides, such as peptides comprising one or more of the extracellular domains, or portions and/or analogs thereof, of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 receptor site, but do not activate the protein, (e.g., receptor-ligand antagonists) can be effective in inhibiting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein activity.

Further, antisense and ribozyme molecules which inhibit expression of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene may also be used in accordance with the invention to inhibit aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene activity. Still further, triple helix molecules may be utilized in inhibiting aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene activity.

The antisense nucleic acid molecules used in the methods of the invention are typically administered to a subject or generated in situ such that they hybridize with or bind to cellular mRNA and/or genomic DNA encoding a 9145, 1725, 31.1, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA transcripts to thereby inhibit translation of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA. A ribozyme having specificity for a 34021, 1720, 1683, 1552, 1682, 1675, 12825, 9952, 5816, 10002 or 1611-encoding nucleic acid can be designed based upon the nucleotide sequence of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 cDNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 49 or 51). For example, a derivative of a Tetrahymena L-19 IVS RNA can be constructed in which the nucleotide sequence of the active site is complementary to the nucleotide sequence to be cleaved in a 34021, 1720, 1683, 1552, 1682, 1675, 12825, 9952, 5816, 10002 or 1611-encoding mRNA (see, for example, Cech et al. U.S. Pat. No. 4,987,071; and Cech et al. U.S. Pat. No. 5,116,742). Alternatively, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA can be used to select a catalytic RNA having a specific ribonuclease activity from a pool of RNA molecules (see, for example, Bartel, D. and Szostak, J. W. (1993) Science 261:1411-1418). 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression can also be inhibited by targeting nucleotide sequences complementary to the regulatory region of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 (e.g., the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 promoter and/or enhancers) to form triple helical structures that prevent transcription of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein and interfere with its activity may also be used to modulate or inhibit 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein function. Such antibodies may be generated using standard techniques described herein, against the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Antibodies that are specific for one or more extracellular domains of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, for example, and that interfere with its activity, are particularly useful in treating AIDS or an HIV-related disorder. Such antibodies are especially efficient because they can access the target domains directly from the bloodstream. Any of the administration techniques described below which are appropriate for peptide administration may be utilized to effectively administer inhibitory target gene antibodies to their site of action.

(ii) Methods for Restoring or Enhancing Target Gene Activity

Genes that cause AIDS or an HIV-related disorder may be underexpressed within BPH and/or UI. Alternatively, the activity of the protein products of such genes may be decreased, leading to the development of AIDS or an HIV-related disorder. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to AIDS or an HIV-related disorder.

Described in this section are methods whereby the level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity may be increased to levels wherein the symptoms of the HIV-related disorder are ameliorated. The level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity may be increased, for example, by either increasing the level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene expression or by increasing the level of active 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein which is present.

For example, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, at a level sufficient to ameliorate at least one symptom of AIDS or an HIV-related disorder may be administered to a patient exhibiting such symptoms. Any of the techniques discussed below may be used for such administration. One of skill in the art will readily know how to determine the concentration of effective, non-toxic doses of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, utilizing techniques such as those described below.

Additionally, RNA sequences encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein may be directly administered to a patient exhibiting AIDS or an HIV-related disorder, at a concentration sufficient to produce a level of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein such that AIDS or an HIV-related disorder are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene, or a portion thereof, that directs the production of a normal 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene sequences into human cells.

Cells, preferably, autologous cells, containing 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expressing gene sequences may then be introduced or reintroduced into the subject at positions which allow for the amelioration of at least one symptom of AIDS or an HIV-related disorder. Such cell replacement techniques may be preferred, for example, when the gene product is a secreted, extracellular gene product.

C. Pharmaceutical Compositions

Another aspect of the invention pertains to methods for treating a subject suffering from a disease. These methods involve administering to a subject an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity (e.g., an agent identified by a screening assay described herein), or a combination of such agents. In another embodiment, the method involves administering to a subject a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or nucleic acid molecule as therapy to compensate for reduced, aberrant, or unwanted 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 expression or activity.

Stimulation of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is desirable in situations in which 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 is abnormally downregulated and/or in which increased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is likely to have a beneficial effect. Likewise, inhibition of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is desirable in situations in which 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 is abnormally upregulated and/or in which decreased 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity is likely to have a beneficial effect.

The agents which modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity (e.g., a fragment of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or an anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity can also be prepared in the form of suppositories (e.g., with conventional suppository bases such as cocoa butter and other glycerides) or retention enemas for rectal delivery.

In one embodiment, the agents that modulate 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10, 000 grams per mole, organic or inorganic compounds having a molecular weight less than about 5, 000 grams per mole, organic or inorganic compounds having a molecular weight less than about 1, 000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is

furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

D. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity, as well as tailoring the dosage and/or therapeutic regimen of treatment with an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 molecule or 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from AIDS or an HIV-related disorder, with an agent which modulates 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity.

V. Recombinant Expression Vectors and Host Cells Used in the Methods of the Invention

The methods of the invention (e.g., the screening assays described herein) include the use of vectors, preferably expression vectors, containing a nucleic acid encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins, mutant forms of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins, fusion proteins, and the like).

The recombinant expression vectors to be used in the methods of the invention can be designed for expression of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins in prokaryotic or eukaryotic cells. For example, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity assays, (e.g., direct assays or competitive assays described in detail below), or to generate antibodies specific for 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins. In a preferred embodiment, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecule of the invention is introduced, e.g., a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecule within a recombinant expression vector or a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Accordingly, the invention further provides methods for producing a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein using the host cells of the invention. In one embodiment, the method comprises culturing the host cell of the invention (into which a recombinant expression vector encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein has been introduced) in a suitable medium such that a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein is produced. In another embodiment, the method further comprises isolating a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein from the medium or the host cell.

VI. Isolated Nucleic Acid Molecules Used in the Methods of the Invention

The methods of the invention include the use of isolated nucleic acid molecules that encode 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins or biologically active portions thereof, as well as nucleic acid fragments sufficient for use as hybridization probes to identify 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-encoding nucleic acid molecules (e.g., 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA) and fragments for use as PCR primers for the amplification or mutation of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, as a hybridization probe, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49 or 51 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, for example, a fragment which can be used as a probe or primer or a fragment encoding a portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, e.g., a biologically active portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49 or 51. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49 or 51.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tm) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, such as by measuring a level of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-encoding nucleic acid in a sample of cells from a subject e.g., detecting 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA levels or determining whether a genomic 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, due to degeneracy of the genetic code and thus encode the same 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52.

The methods of the invention further include the use of allelic variants of human 34021, 1720, 1683, 1552, 1682, 1675, 12825, 9952, 5816, 10002 or 1611, e.g., functional and non-functional allelic variants. Functional allelic variants are naturally occurring amino acid sequence variants of the human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein that maintain a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50 or 52 or substitution, deletion or insertion of non-critical residues in non-critical regions of the protein.

Non-functional allelic variants are naturally occurring amino acid sequence variants of the human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein that do not have a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52, or a substitution, insertion or deletion in critical residues or critical regions of the protein.

The methods of the present invention may further use non-human orthologues of the human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Orthologues of the human 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein are proteins that are isolated from non-human organisms and possess the same 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52) without altering the biological activity, whereas an “essential” amino acid residue is required for biological activity. For example, amino acid residues that are conserved among the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins of the present invention are not likely to be amenable to alteration.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 49 or 51, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein is preferably replaced with another amino acid residue from the same side chain family. Alternatively, in another embodiment, mutations can be introduced randomly along all or part of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 coding sequence, such as by saturation mutagenesis, and the resultant mutants can be screened for 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49 or 51, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 coding strand, or to only a portion thereof. In one embodiment, an antisense nucleic acid molecule is antisense to a “coding region” of the coding strand of a nucleotide sequence encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. The term “noncoding region” refers to 5′ and 3′ sequences which flank the coding region that are not translated into amino acids (also referred to as 5′ and 3′ untranslated regions).

Given the coding strand sequences encoding 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 disclosed herein, antisense nucleic acids of the invention can be designed according to the rules of Watson and Crick base pairing. The antisense nucleic acid molecule can be complementary to the entire coding region of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA, but more preferably is an oligonucleotide which is antisense to only a portion of the coding or noncoding region of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA. For example, the antisense oligonucleotide can be complementary to the region surrounding the translation start site of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2, 2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2, 6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VII. Isolated 9145, 1725, 311, 837, 58305, 156, 14175, 50352, 32678, 5560, 7240, 8865, 12396, 12397, 13644, 19938, 2077, 1735, 1786.10220, 17822, 33945, 43748, 47161, 81982 or 46777 Proteins and Anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 Antibodies Used in the Methods of the Invention

The methods of the invention include the use of isolated 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins, and biologically active portions thereof, as well as polypeptide fragments suitable for use as immunogens to raise anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies. In one embodiment, native 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins can be isolated from cells or tissue sources by an appropriate purification scheme using standard protein purification techniques. In another embodiment, 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins are produced by recombinant DNA techniques. Alternative to recombinant expression, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or polypeptide can be synthesized chemically using standard peptide synthesis techniques.

As used herein, a “biologically active portion” of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein includes a fragment of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein having a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity. Biologically active portions of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein include peptides comprising amino acid sequences sufficiently identical to or derived from the amino acid sequence of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52, which include fewer amino acids than the full length 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins, and exhibit at least one activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Typically, biologically active portions comprise a domain or motif with at least one activity of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein (e.g., the N-terminal region of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein that is believed to be involved in the regulation of apoptotic activity). A biologically active portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be a polypeptide which is, for example, 25, 50, 75, 100, 125, 150, 175, 200, 250, 300 or more amino acids in length. Biologically active portions of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be used as targets for developing agents which modulate a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 activity.

In a preferred embodiment, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52. In other embodiments, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50 or 52, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50 or 52, yet differs in amino acid sequence due to natural allelic variation or mutagenesis, as described in detail in subsection V above. Accordingly, in another embodiment, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50 or 52.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52, having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0 U), using a PAM120 weight residue table, a gap length penalty of 12 and a gap penalty of 4.

The methods of the invention may also use 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 chimeric or fusion proteins. As used herein, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 “chimeric protein” or “fusion protein” comprises a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide operatively linked to a non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide. An “9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide” refers to a polypeptide having an amino acid sequence corresponding to a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 molecule, whereas a “non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide” refers to a polypeptide having an amino acid sequence corresponding to a protein which is not substantially homologous to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, e.g., a protein which is different from the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein and which is derived from the same or a different organism. Within a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion protein the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide can correspond to all or a portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. In a preferred embodiment, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion protein comprises at least one biologically active portion of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. In another preferred embodiment, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion protein comprises at least two biologically active portions of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Within the fusion protein, the term “operatively linked” is intended to indicate that the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide and the non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide are fused in-frame to each other. The non-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide can be fused to the N-terminus or C-terminus of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide.

For example, in one embodiment, the fusion protein is a GST-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion protein in which the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences are fused to the C-terminus of the GST sequences. Such fusion proteins can facilitate the purification of recombinant 34021, 1720, 1683, 1552, 1682, 1675, 12825, 9952, 5816, 10002 or 1611.

In another embodiment, this fusion protein is a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein containing a heterologous signal sequence at its N-terminus. In certain host cells (e.g., mammalian host cells), expression and/or secretion of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be increased through use of a heterologous signal sequence.

The 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion proteins used in the methods of the invention can be incorporated into pharmaceutical compositions and administered to a subject in vivo. The 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion proteins can be used to affect the bioavailability of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate. Use of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fusion proteins may be useful therapeutically for the treatment of disorders caused by, for example, (i) aberrant modification or mutation of a gene encoding a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein; (ii) mis-regulation of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 gene; and

(iii) aberrant post-translational modification of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

Moreover, the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-fusion proteins used in the methods of the invention can be used as immunogens to produce anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies in a subject, to purify 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 ligands and in screening assays to identify molecules which inhibit the interaction of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 substrate.

Preferably, a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-encoding nucleic acid can be cloned into such an expression vector such that the fusion moiety is linked in-frame to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

The present invention also pertains to the use of variants of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins which function as either 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 agonists (mimetics) or as 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antagonists. Variants of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins can be generated by mutagenesis, e.g., discrete point mutation or truncation of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. An agonist of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins can retain substantially the same, or a subset, of the biological activities of the naturally occurring form of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. An antagonist of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can inhibit one or more of the activities of the naturally occurring form of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein by, for example, competitively modulating a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777-mediated activity of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

In one embodiment, variants of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein which function as either 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 agonists (mimetics) or as 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antagonists can be identified by screening combinatorial libraries of mutants, e.g., truncation mutants, of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein for 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein agonist or antagonist activity. In one embodiment, a variegated library of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 variants is generated by combinatorial mutagenesis at the nucleic acid level and is encoded by a variegated gene library. A variegated library of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 variants can be produced by, for example, enzymatically ligating a mixture of synthetic oligonucleotides into gene sequences such that a degenerate set of potential 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences is expressible as individual polypeptides, or alternatively, as a set of larger fusion proteins (e.g., for phage display) containing the set of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences therein. There are a variety of methods which can be used to produce libraries of potential 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein coding sequence can be used to generate a variegated population of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 fragments for screening and subsequent selection of variants of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. In one embodiment, a library of coding sequence fragments can be generated by treating a double stranded PCR fragment of a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies. An isolated 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein, or a portion or fragment thereof, can be used as an immunogen to generate antibodies that bind 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 using standard techniques for polyclonal and monoclonal antibody preparation. A full-length 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein can be used or, alternatively, antigenic peptide fragments of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be used as immunogens. The antigenic peptide of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52, and encompasses an epitope of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 such that an antibody raised against the peptide forms a specific immune complex with the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 that are located on the surface of the protein, e.g., hydrophilic regions, as well as regions with high antigenicity.

A 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 immunogen is typically used to prepare antibodies by immunizing a suitable subject, (e.g., rabbit, goat, mouse, or other mammal) with the immunogen. An appropriate immunogenic preparation can contain, for example, recombinantly expressed 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein or a chemically synthesized 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide. The preparation can further include an adjuvant, such as Freund's complete or incomplete adjuvant, or similar immunostimulatory agent. Immunization of a suitable subject with an immunogenic 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 preparation induces a polyclonal anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 molecules. The term “monoclonal antibody” or “monoclonal antibody composition”, as used herein, refers to a population of antibody molecules that contain only one species of an antigen binding site capable of immunoreacting with a particular epitope of 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. A monoclonal antibody composition thus typically displays a single binding affinity for a particular 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein with which it immunoreacts.

Polyclonal anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies can be prepared as described above by immunizing a suitable subject with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 immunogen. The anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody titer in the immunized subject can be monitored over time by standard techniques, such as with an enzyme linked immunosorbent assay (ELISA) using immobilized 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. If desired, the antibody molecules directed against 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 can be isolated from the mammal (e.g., from the blood) and further purified by well known techniques, such as protein A chromatography to obtain the IgG fraction. At an appropriate time after immunization, e.g., when the anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 immunogen as described above, and the culture supernatants of the resulting hybridoma cells are screened to identify a hybridoma producing a monoclonal antibody that binds 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777.

Any of the many well known protocols used for fusing lymphocytes and immortalized cell lines can be applied for the purpose of generating an anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777, e.g., using a standard ELISA assay.

Alternative to preparing monoclonal antibody-secreting hybridomas, a monoclonal anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody can be identified and isolated by screening a recombinant combinatorial immunoglobulin library (e.g., an antibody phage display library) with 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 to thereby isolate immunoglobulin library members that bind 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184, 187; Taniguchi, M., European Patent Application 171, 496; Morrison et al. European Patent Application 173, 494; Neuberger et al. PCT International Publication No. WO 86/01533; Cabilly et al. U.S. Pat. No. 4,816,567; Cabilly et al. European Patent Application 125, 023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody can be used to detect 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein (e.g., in a cellular lysate or cell supernatant) in order to evaluate the abundance and pattern of expression of the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 protein. Anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using Taqman™ Analysis

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4, 7,2′,7′-tetrachlorofluorescein), JOE (6-carboxy-4, 5-dichloro-2, 7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating AIDS or an HIV-related disorder, comprising:
a) combining a compound to be tested with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide under conditions suitable for binding of the test compound to the polypeptide; and
b) detecting binding of the test compound to the polypeptide to thereby identify a compound which binds to the polypeptide, thereby identifying a compound capable of treating aids or an HIV-related disorder.

2. The method of claim 1, wherein the compound is selected from the group consisting of a small molecule, a peptide or an antibody.

3. The method of claim 1, wherein the polypeptide further comprises heterologous sequences.

4. The method of claim 1, wherein the polypeptide is an isolated polypeptide, a membrane-bound form of an isolated polypeptide or a cell comprising the polypeptide.

5. The method of claim 4, wherein the cell is an AIDS- or HIV-related cell.

6. The method of claim 1, wherein the binding of the test compound to the polypeptide is detected by a method selected from the group consisting of:
a) a competition binding assay;
b) an immunoassay; and
c) a yeast two-hybrid assay.

7. A method for identifying a compound capable of treating AIDS or an HIV-related disorder, comprising:
a) combining a compound to be tested with a host cell expressing a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide under conditions suitable for binding of the test compound to the polypeptide; and
b) detecting binding of the test compound to the polypeptide to thereby identify a compound which binds to the polypeptide, thereby identifying a compound capable of treating AIDS or an HIV-related disorder.

8. The method of claim 7, wherein the compound is selected from the group consisting of a small molecule, a peptide, an antibody or an antisense nucleic acid molecule.

9. The method of claim 7, wherein the polypeptide further comprises heterologous sequences.

10. The method of claim 7, wherein the host cell is an AIDS- or HIV-related cell.

11. The method of claim 7, wherein the binding of the test compound to the polypeptide is detected by a method selected from the group consisting of:
a) a competition binding assay;
b) an immunoassay; and
c) a yeast two-hybrid assay.

12. A method of identifying a subject having AIDS or an HIV-related disorder, or at risk for developing AIDS or an HIV-related disorder comprising:
a) contacting a sample obtained from the subject comprising polypeptides with a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding substance; and
b) detecting the presence of a polypeptide in the sample that binds to the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 binding substance, thereby identifying a subject having AIDS or an HIV-related disorder, or at risk for developing AIDS or an HIV-related disorder.

13. The method of claim 12, wherein the binding substance is an antibody.

14. The method of claim 12, wherein the binding substance is detectably labeled.

15. A method for treating a subject having AIDS or an HIV-related disorder characterized by aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide activity or aberrant 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid expression comprising administering to the subject a 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator, thereby treating said subject having AIDS or an HIV-related disorder.

16. The method of claim 15, wherein the disorder is a disorder associated with but not limited to AIDS or an HIV-related disorder.

17. The method of claim 15, wherein the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator is administered in a pharmaceutically acceptable formulation.

18. The method of claim 15, wherein the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator is capable of modulating 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 polypeptide activity.

19. The method of claim 18, wherein the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator is an anti-9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 antibody.

20. The method of claim 15, wherein the 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 modulator is capable of modulating 9145, 1725, 311, 837, 58305, 156, 14175, 50352, 32678, 5560, 7240, 8865, 12396, 12397, 13644, 19938, 2077, 1735, 1786, 10220, 17822, 33945, 43748, 47161, 81982 or 46777 nucleic acid expression.
